SEQUENCE LISTING (37 sequences)

SEQ ID NO: 1 - protein, 11 aa, synthetic construct
RASENIYSNLA

SEQ ID NO: 2 - protein, 7 aa, synthetic construct
AATNLQS

SEQ ID NO: 3 - protein, 7 aa, synthetic construct
AATNLQD

SEQ ID NO: 4 - protein, 7 aa, synthetic construct
AATSLQS

SEQ ID NO: 5 - protein, 9 aa, synthetic construct
QHLWGTPYT

SEQ ID NO: 6 - protein, 10 aa, synthetic construct
GFSLTSYGLS

SEQ ID NO: 7 - protein, 17 aa, synthetic construct
YIWYDGNTNFHPSLKSR

SEQ ID NO: 8 - protein, 17 aa, synthetic construct
YIWYDGNTNFHSSLKSR

SEQ ID NO: 9 - protein, 17 aa, synthetic construct
YIWYDGNTNFHSPLKSR

SEQ ID NO: 10 - protein, 17 aa, synthetic construct
TEGHYYGSNYGYYALDY

SEQ ID NO: 11 - protein, 107 aa, synthetic construct
DIQMTQSPSSLSASVGDRVTITCRASENIYSNLAWYQQKPGKAPKLLLYAATNLQSGVPSRFSGSGSGTDFTLTISSLQPEDFATYYCQHLWGTPYTFGGGTKLEIK

SEQ ID NO: 12 - protein, 107 aa, synthetic construct
DIQMTQSPSSLSASVGDRVTITCRASENIYSNLAWYQQKQGKAPKLLLYAATNLQDGVPSRFSGSGSGTDYTLTISSLQPEDFATYFCQHLWGTPYTFGQGTKLEIK

SEQ ID NO: 13 - protein, 107 aa, synthetic construct
DIQMTQSPSSLSASVGDRVTITCRASENIYSNLAWYQQKPGKAPKLLIYAATSLQSGVPSRFSGSGSGTDFTLTISSLQPEDFATYYCQHLWGTPYTFGGGTKVEIK

SEQ ID NO: 14 - protein, 125 aa, synthetic construct
QVQLQESGPGLVKPSETLSLTCTVSGFSLTSYGLSWIRQPPGKGLEWIGYIWYDGNTNFHPSLKSRVTISKDTSKNQFSLKLSSVTAADTAVYYCAKTEGHYYGSNYGYYALDYWGQGTSVTVSS

SEQ ID NO: 15 - protein, 125 aa, synthetic construct
QVQLQESGPGLVKPSETLSLTCTVSGFSLTSYGLSWIRQPPGKGLEWIGYIWYDGNTNFHSSLKSRVTISKDTSKSQVSLKLSSVTAADTAVYYCAKTEGHYYGSNYGYYALDYWGQGTLVTVSS

SEQ ID NO: 16 - protein, 125 aa, synthetic construct
QVQLQESGPGLVKPSETLSLTCTVSGFSLTSYGLSWIRQPPGKGLEWIGYIWYDGNTNFHSPLKSRVTISVDTSKNQFSLKLSSVTAADTAVYYCAKTEGHYYGSNYGYYALDYWGQGTLVTVSS

SEQ ID NO: 17 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 19 - protein, 454 aa, synthetic construct
QVQLQESGPGLVKPSETLSLTCTVSGFSLTSYGLSWIRQPPGKGLEWIGYIWYDGNTNFHPSLKSRVTISKDTSKNQFSLKLSSVTAADTAVYYCAKTEGHYYGSNYGYYALDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 454 aa, synthetic construct
QVQLQESGPGLVKPSETLSLTCTVSGFSLTSYGLSWIRQPPGKGLEWIGYIWYDGNTNFHSSLKSRVTISKDTSKSQVSLKLSSVTAADTAVYYCAKTEGHYYGSNYGYYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - protein, 454 aa, synthetic construct
QVQLQESGPGLVKPSETLSLTCTVSGFSLTSYGLSWIRQPPGKGLEWIGYIWYDGNTNFHSPLKSRVTISVDTSKNQFSLKLSSVTAADTAVYYCAKTEGHYYGSNYGYYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNATYRVVSVLTVLHQDWLNGKEYKCKVSNKALPAPIAATISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 214 aa, synthetic construct
DIQMTQSPSSLSASVGDRVTITCRASENIYSNLAWYQQKPGKAPKLLLYAATNLQSGVPSRFSGSGSGTDFTLTISSLQPEDFATYYCQHLWGTPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 214 aa, unidentified
DIQMTQSPSSLSASVGDRVTITCRASENIYSNLAWYQQKQGKAPKLLLYAATNLQDGVPSRFSGSGSGTDYTLTISSLQPEDFATYFCQHLWGTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 214 aa, synthetic construct
DIQMTQSPSSLSASVGDRVTITCRASENIYSNLAWYQQKPGKAPKLLIYAATSLQSGVPSRFSGSGSGTDFTLTISSLQPEDFATYYCQHLWGTPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 11 aa, unidentified
KASQDAGSAVA

SEQ ID NO: 26 - protein, 7 aa, unidentified
WASTRHT

SEQ ID NO: 27 - protein, 9 aa, unidentified
QQYSSYPWT

SEQ ID NO: 28 - protein, 107 aa, unidentified
DIQLTQSPSFLSASVGDRVTITCKASQDAGSAVAWYQQKPGKAPKLLIYWASTRHTGVPSRFSGSGSGTEFTLTISSLQPEDFATYYCQQYSSYPWTFGGGTKLEIK

SEQ ID NO: 29 - protein, 105 aa, unidentified
RTVAAPSVFIFPPSDEQLKSGTASVVCLLNNFYPREAKVQWKVDNALQSGNSQESVTEQDSKDSTYSLSSTLTKADYEKHKVYACEVTHQGLSSPVTKSFNRGEC

SEQ ID NO: 30 - protein, 212 aa, unidentified
DIQLTQSPSFLSASVGDRVTITCKASQDAGSAVAWYQQKPGKAPKLLIYWASTRHTGVPSRFSGSGSGTEFTLTISSLQPEDFATYYCQQYSSYPWTFGGGTKLEIKRTVAAPSVFIFPPSDEQLKSGTASVVCLLNNFYPREAKVQWKVDNALQSGNSQESVTEQDSKDSTYSLSSTLTKADYEKHKVYACEVTHQGLSSPVTKSFNRGEC

SEQ ID NO: 31 - protein, 8 aa, unidentified
GFTFSRYD

SEQ ID NO: 32 - protein, 10 aa, unidentified
ISGGGRYTYY

SEQ ID NO: 33 - protein, 9 aa, unidentified
PYGNYGMDY

SEQ ID NO: 34 - protein, 119 aa, unidentified
EVQLVESGGGLVQPGGSLRLSCAASGFTFSRYDMSWVRQAPGKGLEWVSTISGGGRYTYYPDSVKGRFTISRDNSKNTLYLQMNSLRAEDTAVYYCTSPYGNYGMDYWGQGTSVTVSSA

SEQ ID NO: 35 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 37 - protein, 445 aa, synthetic construct
EVQLVESGGGLVQPGGSLRLSCAASGFTFSRYDMSWVRQAPGKGLEWVSTISGGGRYTYYPDSVKGRFTISRDNSKNTLYLQMNSLRAEDTAVYYCTSPYGNYGMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2026/0308256 A1
(43) Pub. Date: Oct. 8, 2026

(54) COMBINATIONS OF ANTI-CTLA-4 ANTIBODIES AND ANTI-PD-1 ANTIBODIES AND THERAPEUTIC USES THEREOF

(71) Applicants: OncoC4, Inc., Rockville, MD (US); AcroImmune Biopharma Co., Ltd., Nanjing (CN)

(72) Inventors: Yang Liu, Rockville, MD (US); Pan Zheng, Rockville, MD (US); Martin Devenport, Rockville, MD (US); Vadim Tsvetnitsky, Rockville, MD (US); Xianfeng Fang, Nanjing (CN); Qunmin Zhou, Nanjing (CN); Libing Mu, Nanjing (CN)

(21) Appl. No.: 19/479,621

(22) PCT Filed: Apr. 29, 2024
(86) PCT No.: PCT/US2024/026857

§ 371 Date: Oct. 28, 2025

Related U.S. Application Data
(60) Provisional application No. 63/462,839, filed on Apr. 28, 2023.

(51) Int. Cl. C07K 16/28; A61K 39/00; A61K 47/18; A61K 47/22; A61K 47/26; A61P 35/00

(52) U.S. Cl. CPC C07K 16/2818; A61K 47/183; A61K 47/22; A61K 47/26; A61P 35/00; A61K 2039/507; A61K 2039/54; A61K 2039/545

(57) ABSTRACT

Provided herein are combinations of anti-CTLA-4 antibodies and anti-PD-1 antibodies and uses thereof for treating cancer.

### FIELD OF THE INVENTION

The present invention relates to combinations of anti-CTLA-4 and anti-PD-1 antibodies and uses thereof for treating cancer.

### REFERENCE TO SEQUENCE LISTING

This application contains a Sequence Listing that has been submitted in XML format via Patent Center and is hereby incorporated by reference in its entirety. The file, created on Apr. 25, 2024, is named Sequence-Listing-111005-0606-01PC00.xml, and is 37 kb in size.

### BACKGROUND OF THE INVENTION

Cytotoxic T-lymphocyte-associated protein 4 (CTLA-4) interacts with B7-1 (CD80) and B7-2 (CD86) to ensure proper function of regulatory T cells and protect host against autoinflammatory diseases. Anti-CTLA-4 monoclonal antibodies (mAbs), such as Ipilimumab, have demonstrated strong and broad cancer immunotherapeutic effects (CITE) in a variety of preclinical models and in an early clinical study and are now being approved by the US FDA for use in clinical both as monotherapy and as part of combination therapy with Nivolumab (anti-PD-1). But compared with anti-PD-1/PD-L1 antibodies, CTLA-4-targeting in cancer patients has been less successful. Head-to-head comparisons have revealed that response rates of melanoma patients treated with Ipilimumab were consistently lower than that of anti-PD-1 antibody. Thus, while anti-PD-1/PD-L1 antibodies have gained approval for clinical use in rapidly expanding indications, monotherapy with anti-CTLA-4 antibodies have failed in multiple Phase III clinical trials apart from melanoma.

Moreover, CTLA-4 monotherapy has more immunotherapy-related adverse effects (irAEs) than anti-PD-1/PD-L1 therapy. The rate of severe irAE (Grades 3 and 4) reached 73-90% in neo-adjuvant therapy of melanoma patients receiving the combination of Ipilimumab and Nivolumab. The strong irAEs further limit the doses tolerated by cancer patients. Nevertheless, combination with anti-PD-1 resulted in significantly improved response rates and patient survival in multiple types of cancer. Furthermore, anti-CTLA-4 antibodies can induce long-lasting immunity in cancer patients. Therefore, CTLA-4 remains an important immunotherapy target, but major challenges remain in improving both safety and efficacy of anti-CTLA-4 mAbs.

In general, combination of anti-CTLA-4 antibodies and anti-PD-1/PD-L1 antibodies leads to increased anti-tumor activity. The combination of Nivolumab plus Ipilimumab has been proven effective and received FDA approval for the treatment of patients with metastatic melanoma, renal cell carcinoma, or high microsatellite instability (MSI-high) colorectal carcinoma. For example, in the Phase III trial CheckMate-067 the combination ipilimumab plus nivolumab was compared with nivolumab or ipilimumab monotherapy in untreated metastatic melanoma. At a minimum follow-up of 60 months, the median overall survival was more than 60.0 months (median not reached) in the nivolumab-plus-ipilimumab group and 36.9 months in the nivolumab group, as compared with 19.9 months in the ipilimumab group (hazard ratio for death with nivolumab plus ipilimumab vs. ipilimumab, 0.52; hazard ratio for death with nivolumab vs. ipilimumab, 0.63).

However, compared to PD-1 nivolumab monotherapy or CTLA-4 ipilimumab monotherapy, the Ipilimumab-Nivolumab combination is associated with a less favorable safety profile. As expected from the different mechanisms of action of these agents, which act on distinct lymphocyte subtypes and different receptor sites, their concomitant use, such as 3 mg/kg ipilimumab and 1 mg/kg nivolumab, results in both a higher incidence, and a broader spectrum of immune-related adverse events (irAEs). Treatment-related adverse events of Grade 3 or 4 occurred in 23% of the patients in the nivolumab group, 28% of those in the ipilimumab group, and 59% of those in the nivolumab-plus-ipilimumab group, respectively.

The combination Nivolumab 3 mg/kg q2w plus Ipilimumab 1 mg/kg q6w has been tested in advanced/metastatic non-small cell lung cancer (NSCLC) in the CheckMate-227 clinical trial. Among the patients with a PD-L1 expression level of 1% or more, the median overall survival was 17.1 months (95% confidence interval [CI], 15.0 to 20.1) with Nivolumab plus Ipilimumab and 14.9 months (95% CI, 12.7 to 16.7) with chemotherapy (P=0.007), with 2-year overall survival rates of 40.0% and 32.8%, respectively. The median duration of response was 23.2 months with nivolumab plus ipilimumab and 6.2 months with chemotherapy. The overall survival benefit was also observed in patients with a PD-L1 expression level of less than 1%, with a median duration of 17.2 months (95% CI, 12.8 to 22.0) with nivolumab plus ipilimumab and 12.2 months (95% CI, 9.2 to 14.3) with chemotherapy. Among all the patients in the trial, the median overall survival was 17.1 months (95% CI, 15.2 to 19.9) with Nivolumab plus Ipilimumab and 13.9 months (95% CI, 12.2 to 15.1) with chemotherapy. The percentage of patients with grade 3 or 4 treatment-related adverse events in the overall population was 32.8% with nivolumab plus ipilimumab and 36.0% with chemotherapy. The high risk of severe toxicity of the combination of the two antibodies limits clinical development. Accordingly, there is a need in the art for safer combinations of anti-CTLA-4 and anti-PD-1 antibodies.

### SUMMARY OF THE INVENTION

Provided herein is a composition comprising an anti-CTLA-4 antibody and an anti-PD-1 antibody. The anti-CTLA-4 antibody may comprise (i) a light chain variable region comprising a complementarity determining region (CDR) 1 comprising the sequence set forth in SEQ ID NO: 1, a CDR2 comprising the sequence set forth in SEQ ID NO: 3, and a CDR3 comprising the sequence set forth in SEQ ID NO: 5; and (ii) a heavy chain variable region comprising a CDR1 comprising the sequence set forth in SEQ ID NO: 6, a CDR2 comprising the sequence set forth in SEQ ID NO: 9, and a CDR3 comprising the sequence set forth in SEQ ID NO: 10. The anti-CTLA-4 antibody may comprise a light chain variable region comprising the sequence set forth in SEQ ID NO: 12 and a heavy chain variable region comprising the sequence set forth in SEQ ID NO: 16. The anti-CTLA-4 antibody may comprise a light chain comprising the sequence set forth in SEQ ID NO: 23 and a heavy chain comprising the sequence set forth in SEQ ID NO: 21.

The anti-PD-1 antibody may comprise (i) a light chain variable region comprising a CDR1 comprising the sequence set forth in SEQ ID NO: 25, a CDR2 comprising the sequence set forth in SEQ ID NO: 26, and a CDR3 comprising the sequence set forth in SEQ ID NO: 27; and (ii) a heavy chain variable region comprising a CDR1 comprising the sequence set forth in SEQ ID NO: 31, a CDR2 comprising the sequence set forth in SEQ ID NO: 32, and a CDR3 comprising the sequence set forth in SEQ ID NO: 33. The anti-PD-1 antibody may comprise a light chain variable region comprising the sequence set forth in SEQ ID NO: 28 and a heavy chain variable region comprising the sequence set forth in SEQ ID NO: 34. The anti-PD-1 antibody may comprise a heavy chain comprising an IgG4 comprising the sequence set forth in SEQ ID NO: 35 or 36. The anti-PD-1 antibody may comprise a light chain comprising the sequence set forth in SEQ ID NO: 30 and a heavy chain comprising the sequence set forth in SEQ ID NO: 37. The anti-CTLA-4 antibody may comprise a light chain comprising the sequence set forth in SEQ ID NO: 23 and a heavy chain comprising the sequence set forth in SEQ ID NO: 21; and the anti-PD-1 antibody may comprise a light chain comprising the sequence set forth in SEQ ID NO: 30 and a heavy chain comprising the sequence set forth in SEQ ID NO: 37.

The composition may comprise 1-50 mg/mL of each of the anti-CTLA-4 antibody and the anti-PD-1 antibody. The anti-CTLA-4 and the anti-PD-1 antibody may be co-formulated in the same buffer. The composition may comprise 10 mg/mL, or up to 10 mg/mL, of each of the anti-CTLA-4 antibody and the anti-PD-1 antibody. The composition may comprise a pharmaceutically acceptable carrier. The composition may comprise one or more of a histidine buffer, α, α-trehalose dihydrate, polysorbate 80 (PS80), and EDTA. The composition may comprise 5-40 mM histidine buffer, 7.0-10.0% (w/v) α, α-trehalose dihydrate, and 0.01-0.10 (w/v) PS80. The pH of the composition may be 5.5-6.5. The composition may comprise 20 mM histidine buffer, 8.8% (w/v) α, α-trehalose dihydrate, 0.06% (w/v) PS80, and 0.2 mM EDTA·2Na·2H₂O, and the pH of the composition may be 6.0.

Provided herein is a method of treating a cancer in a subject in need thereof, which may comprise administering to the subject the composition. Also provided are use of the composition in the manufacture of a medicament for treating the cancer, and the composition for use in treating the cancer. The cancer may be a tumor. The cancer may be melanoma, non-small cell lung carcinoma (NSCLC), head and neck squamous cell carcinoma (HNSCC), ovarian cancer, endometrial carcinoma, cervical cancer, renal cell carcinoma, bladder cancer, esophageal cancer, gastric cancer, gastroesophageal (GE) junction cancer, colorectal cancer, anal cancer, hepatocellular carcinoma, cancer of a bile duct, adenoid cystic carcinoma (ACC), pancreatic cancer, or triple negative breast cancer (TNBC). The ovarian cancer may be high grade serous ovarian carcinoma, which may be primary peritoneal cancer or fallopian tube cancer. The cancer may be PD-1 resistant.

10 mg/kg of each of the anti-CTLA-4 antibody and the anti-PD-1 antibody may be administered to or be intended to be administered to the subject. The composition may be administered or be intended to be administered to the subject intravenously. The composition may be administered or be intended to be administered once every about 3 weeks.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a schematic representation of an anti-CTLA-4 antibody structure.

FIG. 2 shows the expected disulfide bond structure of an anti-CTLA-4 antibody.

FIG. 3 shows the expected disulfide bond structure of an anti-PD-1 antibody.

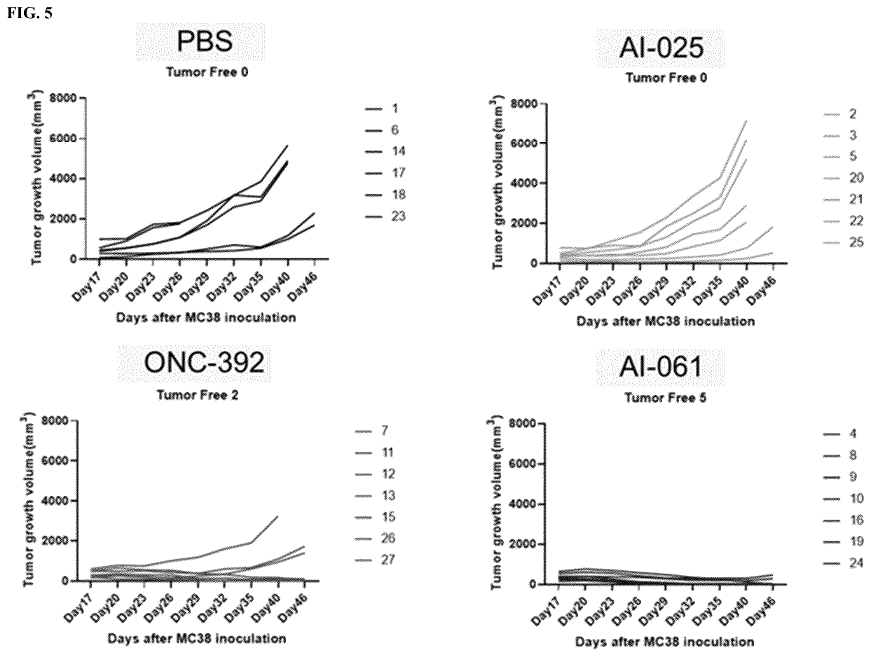

FIG. 4 shows the in vivo effect of anti-PD-1 or anti-CTLA-4 mAb or combination treatment on mouse MC38 tumor in human CTLA4/PD1 double knock-in mice. Human CTLA4/PD1 double knock-in C57BL/6 mice were inoculated with MC38 cells (1×10⁶ cells/mL, 0.1 mL) subcutaneously, when tumor grow to the average volume of about 360 mm³, mice were randomly divided into 7 different groups (n=6, or 7) and received administration of AI-025, ONC-392, or AI-061 at dose of 1.0 mg/kg or PBS (vehicle control) by intraperitoneal injection once every 3 days) for a total of 5 injections. Mice were monitored daily, with the body weight and the size of tumors were measured once every 3 days.

FIG. 5 shows MC38 tumor growth curve in individual mice from different groups. Each line represents the tumor growth from one individual mouse.

FIG. 6 shows a survival curve of the mice represented in FIG. 5.

FIG. 7 shows a drug product manufacturing process flow.

FIG. 8 shows a clinical study design schematic.

FIG. 9A-C show tumor responses in patients treated with 600 mg AI-061. FIG. 9A shows overall response over time, FIG. 9B shows percentage change over time and FIG. 9C shows best percentage change over time.

FIG. 10A-C show tumor responses in patients treated with 400 mg AI-061. FIG. 10A shows overall response over time, FIG. 10B shows percentage change over time and FIG. 10C shows best percentage change over time.

FIG. 11A-C show tumor responses in patients treated with 200 mg AI-061. FIG. 11A shows overall response over time, FIG. 11B shows percentage change over time and FIG. 11C shows best percentage change over time.

FIG. 12 shows mean serum ONC-392 concentration in a PK analysis set.

FIG. 13 shows mean serum AI-025 concentration in a PK analysis set.

### DETAILED DESCRIPTION

Concurrent combination therapy can be achieved by either co-administration of each agent from a conventional single-packaged individual vial or from co-packaged vials, like those of the recently approved Ronapreve [REGEN-COV™, a co-packaged combination of two neutralizing antibodies (Casirivima, IgGIK and Imdevimab, IgG1λ) against the spike protein of the severe acute respiratory syndrome coronavirus 2 (SARS-COV-2)]. Or alternatively, supplied as co-formulated product consisting of a fixed ratio of two or more agents in a same single packaged vial. A co-formulated type of drug product offers many benefits, including reduced risk of doing errors, easier handling, and convenient administration, resulting in improved patient compliance. This is becoming a growing trend in drug development field as the successful introduction of PHESGO® (Pertuzumab, Trastuzumab, and hyaluronidase-zzxf, Roche/Genentech), a subcutaneous co-formulation of two different anti-Her2 antibodies, Pertuzumab and Trastuzumab, with recombinant human hyaluronidase in one ready-to-use, fixed-dose combination vial into the clinical, which has shown acceptable safety, pharmacokinetic and efficacy profiles in clinical trials and gained approval by the US FDA on Jun. 29, 2020 for treating breast cancer.

In keeping with this trend, Bristol Myer Squibb (BMS) has developed OPDUALAG™ which is a fixed dose co-formulation drug comprising of anti-PD-1 mAb Nivolumab and anti-LAG-3 mAb Relatlimab at a fixed ratio of 3:1. OPDUALAG™ was recently approved for melanoma patient based on data from a Phase 3 study. More recently, Merck has initiated Phase 3 study testing MK-1308A (Co-formulated anti-CTLA-4 mAb MK-1308 and anti-PD-1 Pembrolizumab) in participants with advanced clear cell renal cell carcinoma (NCT04736706).

The inventors have had the insight that co-formulating the anti-PD-1 antibody AI-025 and the anti-CTLA-4 antibody ONC-392 would provide a benefit to patients in terms of improved safety and efficacy. This is supported by safety and efficacy data of co-administrating ONC-392 with anti-PD-1 Pembrolizumab. Briefly, in the dose escalation studies involving 13 patients receiving either 3 mg/kg or 6 mg/kg of ONC-392 in combination with a fixed dose of 200 mg Pembrolizumab, the irAE rate was similar between the two dose cohorts (1/7 in the 3 mg/kg and ⅙ in the 6 mg/kg cohorts. Among 10 evaluable patients, a 30% ORR was achieved. By co-formulating the two components, the inventors determined that this will not only reduce the infusion time and avoid dosing errors, but also reduce exposure of non-active components such as PS80. More importantly, in a co-formulation, each of two antibodies maintains it natural structure, and bivalent binding to its targeting antigen. Specifically, in this co-formulation product, AI-025 is a non-depleting humanized IgG4 isotype with high-affinity target binding and ligand blocking activity, while ONC-392 is an Fc-enhanced humanized IgG1 isotype. It has well been demonstrated in preclinical and clinical settings that anti-PD-1 functions solely as an antagonist, whereas ONC-392 efficacy is dependent on depletion of intra-tumoral Tregs.

Finally, the inventors determined that ONC-392 and AI-025 have a similar PK profile in the patients. The PKs of ONC-392 are best described by a 2-compartment model with first-order elimination. The terminal half-life (t_{1/2}) was estimated to be 25.7 days. Baseline albumin was identified as a significant covariate for CL; increased albumin level is associated with decreased CL. Body weight was identified as a significant covariate for volume terms, including central volume (V1) and peripheral volume (V2); increased body weight is associated with increased V1 and V2. No effects of age, sex, race, AST, bilirubin, creatinine clearance, or cancer type on ONC-392 PK were detected. None of these covariates were considered clinically significant. Of note, concurrent chemotherapy with PD-1 was not a significant PK covariate. Preliminary population pharmacokinetics analysis suggests that the PK profile of AI-025 from clinical trial can be characterized by a two-compartment model with first-order elimination. The terminal half-life (t_{1/2}) was estimated to between 9.0-29.2 days.

The inventors have also had the insight to develop AI-061, a co-formulation drug product with a fixed dose (1:1) of ONC-392 and AI-025. This was based on the safety and efficacy profiles of ONC-392 (IgG1 type anti-CTLA4 mAb) and AI-025 (IgG4 type anti-PD-1 mAb) seen in pre-clinical and clinical studies.

ONC-392 is a humanized monoclonal IgG1-kappa isotype antibody against CTLA-4. The parental clone was selected for high anti-tumor efficacy and low autoimmune toxicity. ONC-392 dissociates from CTLA-4 under low pH to allow its escape from lysosomal degradation and recycle to cell surface. In addition, ONC-392 has a modified human IgG1-Fc region to potentially extend its half-life through enhancing binding to FcRn and to increase its ADCC activity by reducing its binding to inhibitory FcRIIB. Therefore, ONC-392 belongs to a new generation of anti-CTLA-4 antibodies that preserves recycling of both CTLA-4 and ONC-392, and thus exhibits minimal irAEs while possessing strong anti-tumor activity. Three lines of evidence support the notion that the pH-sensitive antibody ONC-392 is not only safer but also more effective in Treg depletion and tumor rejection than Ipilimumab, which is pH-insensitive.

First, by preserving CTLA-4 on the cell surface of Tregs in the tumor microenvironment, ONC-392 maintains target density for stronger ADCC activity. In addition, ONC-392 is also recycled after internalization, which allows higher antibody levels in the tumor. As a result of these combined effects, ONC-392 is more efficient in selective depletion of Tregs in tumor microenvironment. Second, ONC-392 is significantly more potent in inducing rejection of large tumors in human CTLA4 knock-in mice. Third, by avoiding down-regulation of CTLA-4 in Tregs outside the tumor microenvironment, ONC-392 preserves the physiological function of CTLA-4 in normal tissues. As a result, ONC-392 causes minimal immune-related adverse events (irAEs) in the CTLA-4 knock-in mouse model that fully recapitulates the irAEs of current clinically used anti-CTLA-4 antibodies. Correspondingly, combination of ONC-392 with anti-PD-1 does not cause overt T cell activation in the peripheral lymphoid organ.

PD-1 interacts with its ligands: PD-L1 (also called as B7-H₁) and PD-L2 (also called as B7-DC) inhibiting or attenuating host immune response to self-tissues and tumors. Compared to CTLA-4, PD-1 has a wider tissue expression pattern, as in addition to activated T and B lymphocytes, other innate immune cells such as NK cells, monocytes and macrophages also showed PD-1 expression either constantly or up-regulated upon stimulation.

The cytoplasmic domain of PD-1 is relatively long with 96 amino acids, which contains both an immunoreceptor tyrosine-based inhibitory motif (ITIM) encompassing a tyrosine at 223 and immunoreceptor tyrosine-based switch motif (ITSM) encompassing a tyrosine at 228. Upon stimulated by TCR/CD3 and CD28 in T cells or engaged by its ligands PD-L1 or PD-L2, these tyrosine residues are phosphorylated, and recruit the Src homology 2-domain-containing tyrosine phosphatase-1 and -2 (SHP-1 and SHP-2) to their sites and then attenuate TCR-mediated T-cell activation and proliferation. Engagement of PD-1 can directly inhibit TcR induced phosphorylation of the ZAP70/CD3zeta signalosome as well as downstream signaling to PKC theta. PD-1 blockade is more effective in attenuating changes in the T-cell transcriptional profile induced by CD3/CD28 co-stimulation than by CTLA-4 blockade, which may be partially explained by recent in-vitro studies data showing that PD-1-bound SHP-2 directly dephosphorylated CD28, demonstrated in a fluorescence resonance energy transfer (FRET)-based assay a cell-free system. In agreement with these in vitro and in vivo pre-clinical study data, clinically approved anti-PD-1 antibodies such as Nivolumab (OPDIVO), Pembrolizumab (KEYTRUDA) or anti-PD-L1 antibodies such as Atezolizumab (TECENTRIQ) have all demonstrated strong and broad cancer immunotherapeutic effects clinically both as monotherapy and as part of combination therapy with Ipilimumab (anti-CTLA-4).

AI-025 is a humanized IgG4-kappa isotype monoclonal antibody against human and monkey PD-1. It has no ADCC or CDC activity as an IgG4 isotype antibody binding weakly to Fc receptors or complements such as Clq. The parental molecule of AI-025 is a mouse monoclonal antibody and did not react to mouse PD-1. Therefore, the non-clinical in-vivo efficacy and on-target toxicity/safety of the AI-025 antibody was mainly tested in a human PD-1 knock-in mouse bearing mouse tumor model. The in-vivo anti-tumor activity of AI-025 has been tested in human PD1 knock-in mice model with mouse colon adenocarcinoma cell line MC38 subcutaneously inoculated. The data showed that AI-025 and OPDIVO (Nivolumab) have a comparable anti-tumor activity. In human PD-1 knock-in mice, the anti-tumor effect is durable and long-lasting with features of immune memory or immune recall response against tumor cell re-challenge.

### 1. Definitions

The terminology used herein is for the purpose of describing particular embodiments only and is not intended to be limiting. As used in the specification and the appended claims, the singular forms “a,” “an” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

### 2. Anti-CTLA-4 Antibodies

Provided herein is an anti-CTLA-4 antibody. The anti-CTLA-4 antibody may be described in U.S. Pat. No. 10,618,960, the contents of which are incorporated herein by reference. The anti-CTLA-4 antibody may comprise a light chain variable region comprising a complementarity determining region (CDR) 1 comprising the amino acid sequence RASENIYSNLA (SEQ ID NO: 1); a CDR2 comprising the amino acid sequence AATNLQS (SEQ ID NO: 2) (LC1), AATNLQD (SEQ ID NO: 3) (LC2), or AATSLQS (SEQ ID NO: 4) (LC3); and, a CDR3 comprising the amino acid sequence QHLWGTPYT (SEQ ID NO: 5).

The light chain variable region comprising one of LC1-LC3 may also comprise one of the following sequences, respectively:

LC1
(SEQ ID NO: 11)
DIQMTQSPSSLSASVGDRVTITCRASENIYSNLAWYQQKPGKAPK
LLLYAATNLQSGVPSRFSGSGSGTDFTLTISSLQPEDFATYYCQH
LWGTPYTFGGGTKLEIK
LC2
(SEQ ID NO: 12)
DIQMTQSPSSLSASVGDRVTITCRASENIYSNLAWYQQKQGKAPK
LLLYAATNLQDGVPSRFSGSGSGTDYTLTISSLQPEDFATYFCQH
LWGTPYTFGQGTKLEIK
LC3
(SEQ ID NO: 13)
DIQMTQSPSSLSASVGDRVTITCRASENIYSNLAWYQQKPGKAPK
LLIYAATSLQSGVPSRFSGSGSGTDFTLTISSLQPEDFATYYCQH
LWGTPYTFGGGTKVEIK
More particularly, the light chain comprising one
of LC1-LC3 may comprise one of the
following amino acid sequences, respectively:
LC1
(SEQ ID NO: 22)
DIQMTQSPSSLSASVGDRVTITCRASENIYSNLAWYQQKPGKAPK
LLLYAATNLQSGVPSRFSGSGSGTDFTLTISSLQPEDFATYYCQH
LWGTPYTFGGGTKLEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC*
LC2
(SEQ ID NO: 23)
DIQMTQSPSSLSASVGDRVTITCRASENIYSNLAWYQQKQGKAPK
LLLYAATNLQDGVPSRFSGSGSGTDYTLTISSLQPEDFATYFCQH
LWGTPYTFGQGTKLEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC*
LC3
(SEQ ID NO: 24)
DIQMTQSPSSLSASVGDRVTITCRASENIYSNLAWYQQKPGKAPK
LLIYAATSLQSGVPSRFSGSGSGTDFTLTISSLQPEDFATYYCQH
LWGTPYTFGGGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC*.

The anti-CTLA-4 antibody may comprise a heavy chain variable region comprising a CDR1 comprising the amino acid sequence GFSLTSYGLS (SEQ ID NO: 6); a CDR2 comprising the amino acid sequence YIWYDGNTNFHPSLKSR (SEQ ID NO: 7) (HC1), YIWYDGNTNFHSSLKSR (SEQ ID NO: 8) (HC2); or, YIWYDGNTNFHSPLKSR (SEQ ID NO: 9) (HC3); and, a CDR3 comprising the amino acid sequence TEGHYYGSNYGYYALDY (SEQ ID NO: 10).

The heavy chain variable regions comprising one of HC1-HC3 may comprise one of the following amino acid sequences, respectively:

HC1
(SEQ ID NO: 14)
QVQLQESGPGLVKPSETLSLTCTVSGFSLTSYGLSWIRQPPGKGL
EWIGYIWYDGNTNFHPSLKSRVTISKDTSKNQFSLKLSSVTAADT
AVYYCAKTEGHYYGSNYGYYALDYWGQGTSVTVSS
HC2
(SEQ ID NO: 15)
QVQLQESGPGLVKPSETLSLTCTVSGFSLTSYGLSWIRQPPGKGL
EWIGYIWYDGNTNFHSSLKSRVTISKDTSKSQVSLKLSSVTAADT
AVYYCAKTEGHYYGSNYGYYALDYWGQGTLVTVSS
HC3
(SEQ ID NO: 16)
QVQLQESGPGLVKPSETLSLTCTVSGFSLTSYGLSWIRQPPGKGL
EWIGYIWYDGNTNFHSPLKSRVTISVDTSKNQFSLKLSSVTAADT
AVYYCAKTEGHYYGSNYGYYALDYWGQGTLVTVSS

The anti-CTLA-4 antibody may comprise a heavy chain constant region from a human Ig protein, which may be IgG, IgE, IgM, IgD, IgA, IgY, IgG1, IgG2, IgG3, IgG4, IgA₁ or IgA₂. In one example, the constant region is a Fc region from a human IgG1 protein. In one example, the heavy chain constant region comprises the amino acid sequence:

(SEQ ID NO: 17)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVMHEAL
HNHYTQKSLSLSPG

The heavy chain constant region may also comprise one or more mutations. Relative to the sequence set forth in SEQ ID NO: 17, the one or more mutations may be selected from M135Y, S137T, T139E, S181A, E216A, and K217A, and a combination thereof. In one example, the heavy chain constant region of the antibody comprises all six mutations. The mutant heavy chain constant region may comprise the amino acid sequence:

(SEQ ID NO: 18)
ASTKGPSVFPLAPSSKSTSGGTAALGCLVKDYFPEPVTVSWNSGA
LTSGVHTFPAVLQSSGLYSLSSVVTVPSSSLGTQTYICNVNHKPS
NTKVDKKVEPKSCDKTHTCPPCPAPELLGGPSVFLFPPKPKDTLY
ITREPEVTCVVVDVSHEDPEVKFNWYVDGVEVHNAKTKPREEQYN
ATYRVVSVLTVLHQDWLNGKEYKCKVSNKALPAPIAATISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVMHEAL
HNHYTQKSLSLSPG

Even more specifically, the heavy chain of the anti-CTLA-4 antibody comprising heavy chain variable regions HC1-HC3 may comprise one of the following amino acid sequences, respectively:

HC1
(SEQ ID NO: 19)
QVQLQESGPGLVKPSETLSLTCTVSGFSLTSYGLSWIRQPPGKGL
EWIGYIWYDGNTNFHPSLKSRVTISKDTSKNQFSLKLSSVTAADT
AVYYCAKTEGHYYGSNYGYYALDYWGQGTSVTVSSASTKGPSVFP
LAPSSKSTSGGTAALGCLVKDYFPEPVTVSWNSGALTSGVHTFPA
VLQSSGLYSLSSVVTVPSSSLGTQTYICNVNHKPSNTKVDKKVEP
KSCDKTHTCPPCPAPELLGGPSVFLFPPKPKDTLYITREPEVTCV
VVDVSHEDPEVKFNWYVDGVEVHNAKTKPREEQYNATYRVVSVLT
VLHQDWLNGKEYKCKVSNKALPAPIAATISKAKGQPREPQVYTLP
PSRDELTKNQVSLTCLVKGFYPSDIAVEWESNGQPENNYKTTPPV
LDSDGSFFLYSKLTVDKSRWQQGNVFSCSVMHEALHNHYTQKSLS
LSPG**
HC2
(SEQ ID NO: 20)
QVQLQESGPGLVKPSETLSLTCTVSGFSLTSYGLSWIRQPPGKGL
EWIGYIWYDGNTNFHSSLKSRVTISKDTSKSQVSLKLSSVTAADT
AVYYCAKTEGHYYGSNYGYYALDYWGQGTLVTVSSASTKGPSVFP
LAPSSKSTSGGTAALGCLVKDYFPEPVTVSWNSGALTSGVHTFPA
VLQSSGLYSLSSVVTVPSSSLGTQTYICNVNHKPSNTKVDKKVEP
KSCDKTHTCPPCPAPELLGGPSVFLFPPKPKDTLYITREPEVTCV
VVDVSHEDPEVKFNWYVDGVEVHNAKTKPREEQYNATYRVVSVLT
VLHQDWLNGKEYKCKVSNKALPAPIAATISKAKGQPREPQVYTLP
PSRDELTKNQVSLTCLVKGFYPSDIAVEWESNGQPENNYKTTPPV
LDSDGSFFLYSKLTVDKSRWQQGNVFSCSVMHEALHNHYTQKSLS
LSPG**
HC3
(SEQ ID NO: 21)
QVQLQESGPGLVKPSETLSLTCTVSGFSLTSYGLSWIRQPPGKGL
EWIGYIWYDGNTNFHSPLKSRVTISVDTSKNQFSLKLSSVTAADT
AVYYCAKTEGHYYGSNYGYYALDYWGQGTLVTVSSASTKGPSVFP
LAPSSKSTSGGTAALGCLVKDYFPEPVTVSWNSGALTSGVHTFPA
VLQSSGLYSLSSVVTVPSSSLGTQTYICNVNHKPSNTKVDKKVEP
KSCDKTHTCPPCPAPELLGGPSVFLFPPKPKDTLYITREPEVTCV
VVDVSHEDPEVKFNWYVDGVEVHNAKTKPREEQYNATYRVVSVLT
VLHQDWLNGKEYKCKVSNKALPAPIAATISKAKGQPREPQVYTLP
PSRDELTKNQVSLTCLVKGFYPSDIAVEWESNGQPENNYKTTPPV
LDSDGSFFLYSKLTVDKSRWQQGNVFSCSVMHEALHNHYTQKSLS
LSPG**

A C-terminal lysine (K) may be additionally included in the amino acid sequence of the heavy chains set forth in SEQ ID NOs: 19-21, which may increase expression levels. The terminal lysine may be cleaved naturally during production of the anti-CTLA-4 antibody, or upon administration of the antibody.

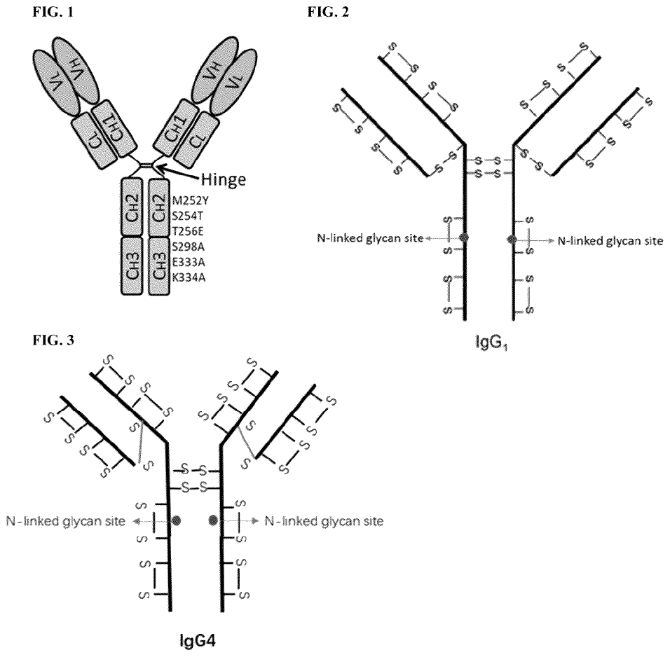

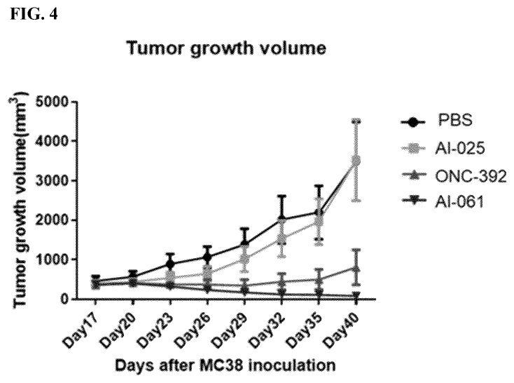

PP4637 (LC2/HC3): In one example the anti-CTLA-4 antibody comprises a light chain variable region comprising a CDR1 comprising the sequence set forth in SEQ ID NO: 1, a CDR2 comprising the sequence set forth in SEQ ID NO: 3, and a CDR3 comprising the sequence set forth in SEQ ID NO: 5. The heavy chain variable region comprises a CDR1 comprising the sequence set forth in SEQ ID NO: 6, a CDR2 comprising the sequence set forth in SEQ ID NO: 9, and a CDR3 comprising the sequence set forth in SEQ ID NO: 10. In particular, the light chain variable region may comprise the sequence set forth in SEQ ID NO: 12 and the heavy chain variable region may comprise the sequence set forth in SEQ ID NO: 16. More particularly, the light chain may comprise the sequence set forth in SEQ ID NO: 23, and the heavy chain may comprise the sequence set forth in SEQ ID NO: 21. This antibody may be referred to as ONC-392. The structure of anti-CTLA-4 antibody ONC-392 is shown in FIG. 1. A schematic of expected disulfide bond structure of ONC-392 is shown in FIG. 2.

PP4631 (LC2/HC1): In another example, the anti-CTLA-4 antibody comprises a light chain variable region comprising a CDR1 comprising the sequence set forth in SEQ ID NO: 1, a CDR2 comprising the sequence set forth in SEQ ID NO: 3, and a CDR3 comprising the sequence set forth in SEQ ID NO: 5. The heavy chain variable region comprises a CDR1 comprising the sequence set forth in SEQ ID NO: 6, a CDR2 comprising the sequence set forth in SEQ ID NO: 7, and a CDR3 comprising the sequence set forth in SEQ ID NO: 10. In particular, the light chain variable region may comprise the sequence set forth in SEQ ID NO: 13 and the heavy chain variable region may comprise the sequence set forth in SEQ ID NO: 14. More particularly, the light chain may comprise the sequence set forth in SEQ ID NO: 23, and the heavy chain may comprise the sequence set forth in SEQ ID NO: 19.

PP4638 (LC3/HC3): In a further example, the anti-CTLA-4 antibody comprises a light chain variable region comprising a CDR1 comprising the sequence set forth in SEQ ID NO: 1, a CDR2 comprising the sequence set forth in SEQ ID NO: 4, and a CDR3 comprising the sequence set forth in SEQ ID NO: 5. The heavy chain variable region comprises a CDR1 comprising the sequence set forth in SEQ ID NO: 6, a CDR2 comprising the sequence set forth in SEQ ID NO: 9, and a CDR3 comprising the sequence set forth in SEQ ID NO: 10. In particular, the light chain variable region may comprise the sequence set forth in SEQ ID NO: 12 and the heavy chain variable region may comprise the sequence set forth in SEQ ID NO: 16. More particularly, the light chain may comprise the sequence set forth in SEQ ID NO: 24, and the heavy chain may comprise the sequence set forth in SEQ ID NO: 21.

### 3. Anti-PD-1 Antibodies

Provided herein is an anti-PD-1 antibody. The anti-PD-1 antibody may be described in U.S. Pat. No. 11,345,754. The anti-PD-1 antibody may comprise a light chain comprising a light chain variable region, which may comprise one or more of a complementarity determining region (CDR) 1 comprising the sequence KASQDAGSAVA (SEQ ID NO: 25), a CDR2 comprising the sequence WASTRHT (SEQ ID NO: 26), and a CDR3 comprising the sequence QQYSSYPWT (SEQ ID NO: 27). The light chain variable region may comprise the following sequence.

(SEQ ID NO: 28)
DIQLTQSPSFLSASVGDRVTITCKASQDAGSAVAWYQQKPGKAPK
LLIYWASTRHTGVPSRFSGSGSGTEFTLTISSLQPEDFATYYCQQ
YSSYPWTFGGGTKLEIK

The light chain may comprise a constant region comprising the following sequence.

(SEQ ID NO: 29)
RTVAAPSVFIFPPSDEQLKSGTASVVCLLNNFYPREAKVQWKVDN
ALQSGNSQESVTEQDSKDSTYSLSSTLTKADYEKHKVYACEVTHQ
GLSSPVTKSFNRGEC

The light chain may comprise the following sequence.

(SEQ ID NO: 30)
DIQLTQSPSFLSASVGDRVTITCKASQDAGSAVAWYQQKPGKAPK
LLIYWASTRHTGVPSRFSGSGSGTEFTLTISSLQPEDFATYYCQQ
YSSYPWTFGGGTKLEIKRTVAAPSVFIFPPSDEQLKSGTASVVCL
LNNFYPREAKVQWKVDNALQSGNSQESVTEQDSKDSTYSLSSTLT
KADYEKHKVYACEVTHQGLSSPVTKSFNRGEC

The anti-PD-1 antibody may comprise a heavy chain comprising a heavy chain variable region, which may comprise one or more of a CDR1 comprising the sequence GFTFSRYD (SEQ ID NO: 31), a CDR2 comprising the sequence ISGGGRYTYY (SEQ ID NO: 32), and a CDR3 comprising the sequence PYGNYGMDY (SEQ ID NO: 33). The heavy chain variable region may comprise the following sequence.

(SEQ ID NO: 34)
EVQLVESGGGLVQPGGSLRLSCAASGFTFSRYDMSWVRQAPGKGL
EWVSTISGGGRYTYYPDSVKGRFTISRDNSKNTLYLQMNSLRAED
TAVYYCTSPYGNYGMDYWGQGTSVTVSSA

The heavy chain may comprise a constant region, which may be a wild-type IgG4 constant region comprising the following sequence.

(SEQ ID NO: 35)
ASTKGPSVFPLAPCSRSTSESTAALGCLVKDYFPEPVTVSWNSGA
LTSGVHTFPAVLQSSGLYSLSSVVTVPSSSLGTKTYTCNVDHKPS
NTKVDKRVESKYGPPCPSCPAPEFLGGPSVFLFPPKPKDTLMISR
TPEVTCVVVDVSQEDPEVQFNWYVDGVEVHNAKTKPREEQFNSTY
RVVSVLTVLHQDWLNGKEYKCKVSNKGLPSSIEKTISKAKGQPRE
PQVYTLPPSQEEMTKNQVSLTCLVKGFYPSDIAVEWESNGQPENN
YKTTPPVLDSDGSFFLYSRLTVDKSRWQEGNVFSCSVMHEALHNH
YTQKSLSLSLGK

The IgG4 heavy chain may be a mutant IgG4 heavy chain comprising a serine to proline change at position 226 of the antibody, which may be in the hinge region. The mutant IgG4 constant region may comprise the following sequence.

(SEQ ID NO: 36)
ASTKGPSVFPLAPCSRSTSESTAALGCLVKDYFPEPVTVSWNSGA
LTSGVHTFPAVLQSSGLYSLSSVVTVPSSSLGTKTYTCNVDHKPS
NTKVDKRVESKYGPPCPPCPAPEFLGGPSVFLFPPKPKDTLMISR
TPEVTCVVVDVSQEDPEVQFNWYVDGVEVHNAKTKPREEQFNSTY
RVVSVLTVLHQDWLNGKEYKCKVSNKGLPSSIEKTISKAKGQPRE
PQVYTLPPSQEEMTKNQVSLTCLVKGFYPSDIAVEWESNGQPENN
YKTTPPVLDSDGSFFLYSRLTVDKSRWQEGNVFSCSVMHEALHNH
YTQKSLSLSLGK

The heavy chain may comprise the following sequence.

(SEQ ID NO: 37)
EVQLVESGGGLVQPGGSLRLSCAASGFTFSRYDMSWVRQAPGKGL
EWVSTISGGGRYTYYPDSVKGRFTISRDNSKNTLYLQMNSLRAED
TAVYYCTSPYGNYGMDYWGQGTSVTVSSASTKGPSVFPLAPCSRS
TSESTAALGCLVKDYFPEPVTVSWNSGALTSGVHTFPAVLQSSGL
YSLSSVVTVPSSSLGTKTYTCNVDHKPSNTKVDKRVESKYGPPCP
PCPAPEFLGGPSVFLFPPKPKDTLMISRTPEVTCVVVDVSQEDPE
VQFNWYVDGVEVHNAKTKPREEQFNSTYRVVSVLTVLHQDWLNGK
EYKCKVSNKGLPSSIEKTISKAKGQPREPQVYTLPPSQEEMTKNQ
VSLTCLVKGFYPSDIAVEWESNGQPENNYKTTPPVLDSDGSFFLY
SRLTVDKSRWQEGNVFSCSVMHEALHNHYTQKSLSLSLGK

The anti-PD-1 antibody may comprise the light chain comprising the sequence set forth in SEQ ID NO: 30 and a heavy chain comprising the sequence set forth in SEQ ID NO: 37. This anti-PD-1 antibody may be referred to herein as AI-025. A schematic of expected disulfide bond structure of anti-PD-1 antibody AI-025 is shown in FIG. 2.

The CDRs of the heavy chain of mAb21 may have been transplanted into the frame region of the human heavy chain variable region (VH) from IgHV3-23 (NCBI Gene ID: 28442), which may have the highest protein sequence homology to AI-025 heavy chain V-region. The CDRs of the light chain of mAb21 may have been transplanted into the frame region or human light chain variable region (VL) from IgKV1-9 (NCBI Gene ID: 28941), which may have the highest protein sequence homology to AI-025 light chain V-region.

The humanized VH may be linked with the human IgG4 heavy chain constant region (CH) (UniProtKB/Swiss-Prot: P01861.1) with a mutation of the serine residue at position 226 in the hinge region to the proline residue (S226P), and the humanized VL may be linked to human kappa light chain constant region (UniProtKB/Swiss-Prot: P01834.2), thereby generating a fully humanized AI-025 antibody of IgG4/kappa type. The IgG4 heavy chain may reduce the potential of in vivo antibody-dependent cellular cytotoxicity (ADCC) or complement-directed cytotoxicity (CDC) effects on PD-1 positive immune cells such as antigen activated T-lymphocytes, and the S226P mutation in the hinge region may prevent the formation of IgG4 half-chain molecule and Fab arm exchange.

### 4. Combined Antibody Compositions

Provided herein is a composition comprising a co-formulation of at least one of the anti-CTLA-4 antibody and at least one of the anti-PD-1 antibody. The composition may also comprise a pharmaceutically acceptable carrier. In one example, the anti-CTLA-4 antibody is ONC-392 and the anti-PD-1 antibody is AI-025. The composition comprising the combination of ONC-392 and AI-025 may be referred to as AI-061. AI-061 may comprise an equal amount of ONC-392 and AI-025. AI-061 may be a potent immune-check point inhibitor due to its nature of dual-targeting of CTLA-4 and PD-1. The anti-CTLA-4 antibody and the anti-PD-1 antibody may be co-formulated in the same composition, which may comprise a buffer a disclosed herein.

The composition may comprise a sterile solution for intravenous infusion. The composition may be a preservative-free aqueous solution, and may be suitable for parenteral administration. In one example, the composition independently comprises about 5, 10, 20, 30, 40, or 50 mg/mL, or an amount in a range of two of these amounts, of each of the anti-CTLA-4 antibody and the anti-PD-1 antibody. The composition may comprise about 5, 10, or 20 mg/mL of each of the anti-CTLA-4 antibody and the anti-PD-1 antibody. The composition may comprise an anti-CTLA-4 antibody-to-anti-PD-1 antibody ratio of about 10:1, 5:1, 4:1, 3:1, 2:1, 4:3, 3:2, 1:1, 2:3, 3:4, 1:2, 1:3, 1:4, 1:5, or 1:10. In one example, the composition comprises 10 mg/ml of each of the anti-CTLA-4 antibody and the anti-PD-1 antibody. In one example, the composition comprises 100 mg of each of the anti-CTLA-4 antibody and the anti-PD-1 antibody. The composition may comprise about 5, 10, 15, 20, 25, or 30 mL of an aqueous solution of the antibodies. The aqueous solution may be contained in a vial. The volume of the aqueous solution in the vial may be 10 mL. In one example, the anti-CTLA-4 and anti-PD-1 antibodies are present at a combined protein concentration of 20 mg/mL.

The pharmaceutically acceptable carrier may comprise one or more of a histidine buffer, an acetate buffer, trehalose, PS80, sucrose, and EDTA. The composition may comprise about 5, 10, 15, 20, 25, 30, 35, or 40 mM histidine buffer, or an amount in a range of two of these amounts. In one example, the composition comprises 20 mM histidine buffer. The composition may also comprise about 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0% (w/v) α, α-trehalose dihydrate, or an amount in a range of two of these amounts. In one example, the composition comprises 8.8% (w/v) α, α-trehalose dihydrate. The composition may comprise about 0.01, 0.02, 0.03, 0.04, 0.05, 0.06, 0.07, 0.08, 0.09, or 0.10 (w/v) PS80, or an amount in a range of two of these amounts. In one example, the composition comprises 0.06% (w/v) PS80. The composition may comprise about 0.05, 0.10, 0.15, 0.20, 0.25, 0.30, 0.35, or 0.40 mM EDTA, which may be EDTA·2Na·2H₂O. In one example, the composition comprises 0.2 mM EDTA. The composition may have a pH of 5.5, 5.6, 5.7, 5.8, 5.9, 6.0, 6.1, 6.2, 6.3, 6.4, or 6.5, or a pH in a range thereof.

In one example, the composition comprises 20 mM histidine buffer, 8.8% (w/v) α, α-trehalose dihydrate, and 0.06% (w/v) PS80, at a pH of 6.0. In another example, the composition further comprises 0.2 mM EDTA·2Na·2H₂O. The composition may comprise 20 mM acetate buffer, 9% (w/v) sucrose, and 0.02% (w/v) PS80, and may have a pH of 5.8.

Each vial may be filled with an extractable volume of 10 mL for a total of approximately 100 mg/vial of ONC-392 and 100 mg/vial of AI-025, respectively.

### 5. Dosing Regimens

The anti-CTLA-4 antibody and the anti-PD-1 antibody may be administered systemically, which may be via injection or intravenous (IV) administration. One or more doses of the anti-CTLA-4 antibody and the anti-PD-1 antibody may be administered to or be intended for administration to a subject. Independently, each dose for each antibody may be about 0.1 mg/kg, 0.3 mg/kg, 1 mg/kg, 3 mg/kg, 5, mg/kg, 6 mg/kg, 10 mg/kg, 15 mg/kg, or 20 mg/kg, or an amount in a range of two of these amounts. Each dose of each antibody may be about 50, 100, 150, 200, 250, 300, 350, 400, 450, 500, 550, 600, 650, 700, 750, 800, 850, 900, 950, 1000 mg, or an amount in a range of two of these amounts, for a subject with a body weight of 30 kg or higher. Each dose of each antibody may be about 50-1000 mg for a subject with a body weight of 30 kg or higher. The doses of the anti-CTLA-4 antibody and the anti-PD-1 antibody administered may be at a ratio of anti-CTLA-4 antibody-to-anti-PD-1 antibody of about 10:1, 5:1, 4:1, 3:1, 2:1, 4:3, 3:2, 1:1, 2:3, 3:4, 1:2, 1:3, 1:4, 1:5, or 1:10. In one example, the anti-CTLA-4 antibody and the anti-PD-1 antibody are combined in a single formulation at a 1:1 ratio, where each antibody is present at an amount of 100, 200, or 300 mg (200, 400, or 600 mg total antibody).

When the anti-CTLA-4 antibody and the anti-PD-1 antibody are each at a dose of 100 mg, then they may be infused or intended to be infused over a period of at least 30 min. When the anti-CTLA-4 antibody and the anti-PD-1 antibody are each at a dose of 200 mg or more, then they may be infused or intended to be infused over a period of at least 60 min.

The dose of each anti-CTLA-4 antibody and anti-PD-1 antibody may independently be about 1, 2, 3, 6, 10, 15, or 20 mg/kg, or an amount in a range of two of these amounts. The dose may comprise 1-20 mg/kg of each of the anti-CTLA-4 antibody and the anti-PD-1 antibody. The dose may be 50-1000 mg of each of the anti-CTLA-4 antibody and the anti-PD-1 antibody. The body weight of the subject may be at least 30 kg.

The antibodies may be administered periodically, each administration for which one of the foregoing doses is administered to the subject. At each cycle of dosing, the dose may be different from a previous dose. The dosing may involve escalating doses. In one example, the antibodies are administered about every 1, 2, 3, 4, 5, or 6 weeks. In particular, the antibodies are administered about every 3 weeks. When describing the period of a dosing cycle, “about” may mean±1, 2, or 3 days.

In one example, the dose of each of the anti-CTLA-4 antibody and the anti-PD-1 antibody is 10 mg/kg. The anti-CTLA-4 antibody and the anti-PD-1 antibody may be administered in a dosing regimen comprising 10 mg/kg for two doses, followed by 1-6 mg/kg for extended dosing (that is, each subsequent dose is 1-6 mg/kg). The extended dosing may comprise administering a dose of 3 mg/kg or 6 mg/kg. In one example, each administration is once about every 3 weeks. In one example, the anti-CTLA-4 antibody and the anti-PD-1 antibody are administered once every about 4 weeks. The dosing may take place over a period of about 3, 6, 9, 12, 15, 18, 21, 24, 27, 30, 33, 36, 39, 42, 45, 48, 51, or 52 weeks, or within a range thereof.

Body weight was shown not to be a significant covariant for clearance of ONC-392. The clinical studies demonstrate that ONC-392 as a single agent or combined with an anti-PD-1 antibody was well tolerated at doses up to 10 mg/kg in monotherapy or 6 mg/kg when used in combination with 200 mg flat dose of pembrolizumab in cancer patients. Therefore, a flat dose of each of the anti-CTLA-4 antibody and the anti-PD-1 antibody may be administered, which may comprise about 100 mg, 200 mg (~1.5 mg/kg each), 400 mg (~3 mg/kg each), 600 mg (~4.5 mg/kg each), 800 mg (~6.0 mg/kg each), or 1000 mg (~7.5 mg/kg each) of each antibody, or an amount in a range of two of these amounts.

### 6. Methods of Treatment

Provided herein is a method of treating a cancer in a subject, who may be in need thereof. The method may comprise administering to the subject a combination of at least one anti-CTLA-4 antibody and at least one anti-PD-1 antibody disclosed herein to the subject. In one example, the anti-CTLA-4 antibody is ONC-392 and the anti-PD-1 antibody is AI-025. In a further example, the combination of the anti-CTLA-4 antibody and the anti-PD-1 antibody is AI-061. Further provided herein are use of the anti-CTLA-4 antibody/anti-PD-1 antibody combination in the manufacture of a medicament for treating a cancer, and the combination for use in treating a cancer.

The cancer may be a carcinoma, including that of the bladder, breast, colon, kidney, liver, lung, ovary, pancreas, stomach, cervix, head and neck squamous cell carcinoma (HNSCC), endometrial, renal cell, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Berketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xenoderma pegmentosum, keratoactanthoma, seminoma, thyroid follicular cancer and teratocarcinoma. It is also contemplated that cancers caused by aberrations in apoptosis would also be treated by the methods and compositions of the invention. Such cancers may include, but are not be limited to, follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated or prevented by the methods and compositions disclosed herein, including of the ovary, bladder, esophagous, gastrointestinal region, liver, bile duct, adenoids, breast, colon, lung, skin, pancreas, or uterus. In other specific embodiments, sarcoma, melanoma, or leukemia is treated or prevented by the methods and compositions of the invention.

In one example, the cancer is melanoma, non-small cell lung carcinoma (NSCLC), HNSCC, ovarian cancer, endometrial carcinoma, cervical cancer, renal cell carcinoma, bladder cancer, esophageal cancer, gastric cancer, gastroesophageal (GE) junction cancer, colorectal cancer, anal cancer, hepatocellular carcinoma, cancer of a bile duct (e.g., cholangiocarcinoma), adenoid cystic carcinoma (ACC), pancreatic cancer, or triple negative breast cancer (TNBC). In a further example, the NSCLC is PD-1-resistant. The ovarian cancer may be high grade serous ovarian carcinoma, which may be primary peritoneal cancer or fallopian tube cancer. The TNBC may be PD-1 resistant. The melanoma may be immuno-oncology (IO)-resistant.

In one example the cancer is a solid tumor. The solid tumor may have been one or more of histologically confirmed and cytologically confirmed. The tumor may be in a subject who has progressive locally advanced or metastatic disease. The subject may have exhibited failure or intolerance to at least one established standard medical anti-cancer therapy. The subject's cancer diagnosis may be determined by a standard of practice, which may be National Comprehensive Cancer Network (NCCN) Clinical Practice Guidelines in Oncology.

The present invention has multiple aspects, illustrated by the following non-limiting examples.

### Example 1

### Combined Anti-CTLA-4 Antibody and Anti-PD-1 Antibody

The in vivo anti-tumor activity of AI-061 was tested in human CTLA4 and PD1 double knock-in mice in a model of subcutaneously inoculated mouse colon adenocarcinoma cell line MC38, which is sensitivity to PD-1 and CTLA-4 therapy. This study was conducted with single agent ONC-392 (anti-CTLA-4) or AI-025 (anti-PD-1) included as the control for a comparison. Table 1 shows a summary of mouse tumor model, treatment group and schedule.

Briefly, human CTLA4/PD1 double knock-in C57BL/6 mice (6-8 weeks old) were inoculated with MC38 cells (1×10⁶ cells/mL, 0.1 mL) subcutaneously, when tumor grow to approximately 360 mm³, mice were randomly divided into 4 different groups (n=6 or 7) and received the administration of the phosphate buffer saline (PBS, vehicle control), or AI-025 (anti-PD-1), ONC-392 (anti-CTLA-4), and AI-061 (anti-CTLA-4 plus anti-PD-1), all at dose level 1 mg/kg body weight by intraperitoneal injection once every 3 days for a total of 5 doses.

TABLE 1
Summary of mouse tumor model, treatment group and schedule

| | | | |
|---|---|---|---|
| | Dose | Number | |
| Group | (mg/kg) | of mice | Mice ID |

| | | |
|---|---|---|
| Group 1: PBS (vehicle control) | 0 | 6 |
| Group 2: AI-025 (anti-PD-1) | 1 | 7 |
| Group 3: ONC-392 (anti-CTLA-4) | 1 | 7 |
| Group 4: AI-061 | 1 | 7 |
| (anti-PD-1 plus anti-CTLA-4) | | |

| | |
|---|---|
| Tumor model | Mouse MC38 colon adenocarcinoma cells subcutaneously |
| | inoculated into human CTLA4/PD1 double knock-in |
| | C57BL/6 mice, when tumor reached approximately 360 |
| | mm3, mice randomly divided into 7 groups (n = 6 or 7) |
| Treatment Schedule | intraperitoneal injection (i.p.) once every 3 days, for a total |
| | of 5 injections |

Mice were observed daily for survival and sign of toxicity (mouse body weight was used as a reference index for indirect determination of drug toxicity), and tumor volume (V) was measured every 3 days (length× width) with a caliper. The tumor volume (V)=0.5a×b², whereas the a is the long diameter (length) and b is the short diameter (width) of tumor.

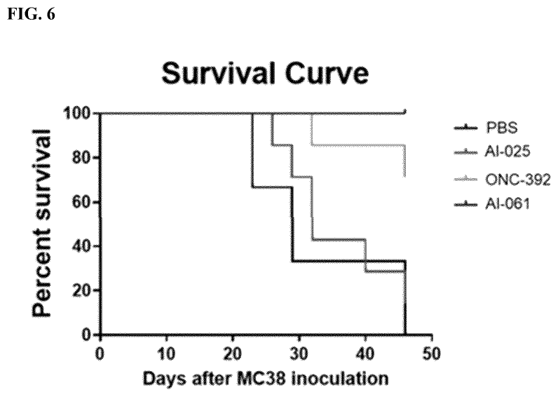

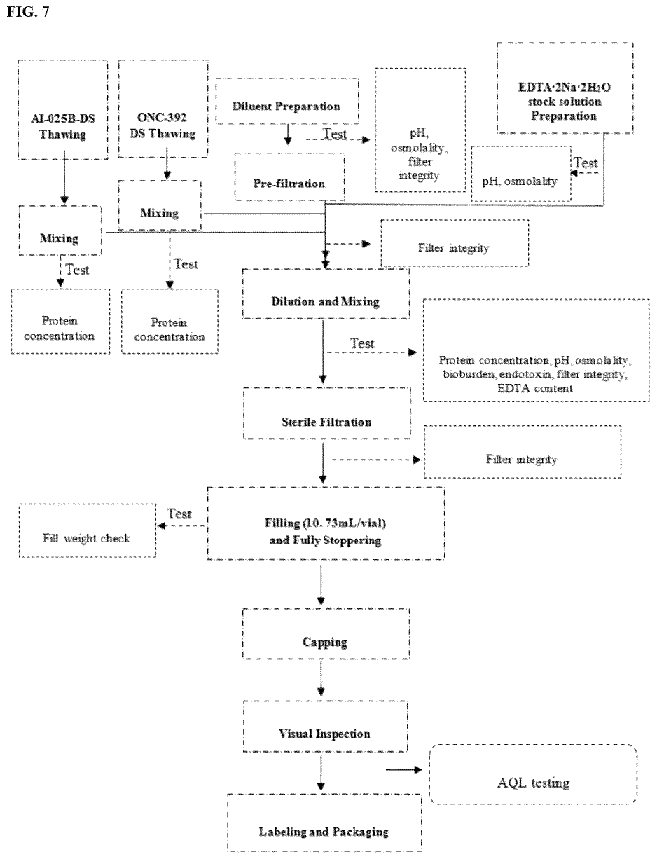

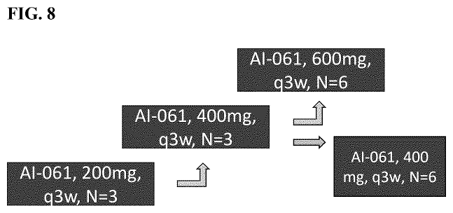

The mean tumor volume at different days after the treatment in each group are shown in Table 2, and the tumor growth curve in each group (mean±SEM) or in each individual mouse were shown in FIGS. 4 and 5, respectively. As show in the below Table and Figures, at an equal dose level of 1.0 mg/kg, AI-061 treatment had a superior anti-tumor activity as compared to treatment with either ONC-392 or AI-025 alone. At the end of the study (Day 46 after the treatment), 5 of 7 mice in AI-061 treatment group were tumor free, whereas in the PBS vehicle, AI-025 or ONC-392 treatment group, there were 0 of 6, 0 of 7, or 2 of 7 mice were tumor free. AI-061 treatment also appears safe, with a 100% mouse survival rate (FIG. 6).

TABLE 2
Mean tumor volume at different days
after the treatment in each group

Tumor volume (mm³) ^{a}

| | | AI-025 | ONC-392 | AI-061 |
|---|---|---|---|---|
| Days | Vehicle | 1 mg/kg | 1 mg/kg | 1 mg/kg |
| 0 ^{b} | 464.27 | 406.77 | 360.66 | 377.19 |
| 3 | 574.92 | 444.28 | 418.92 | 403.78 |
| 6 | 894.98 | 543.82 | 378.46 | 326.97 |
| 9 | 1070.54 | 647.05 | 376.90 | 239.01 |
| 12 | 1385.24 | 1018.96 | 346.94 | 177.81 |
| 15 | 2013.83 | 1536.08 | 449.06 | 125.66 |
| 18 | 2197.71 | 1964.79 | 503.38 | 114.72 |
| 21 | 3498.93 | 3526.04 | 810.97 | 85.47 |

^{a} mean values in each group (n = 6 or 7),
^{b} Days after mice received the first treatment by i.p.

### Example 2

### Formulation Development

This example demonstrates the development of the formulation for an antibody composition disclosed herein. The development included studies designed to select a buffer system, excipients, surfactant and an antioxidant to stabilize the protein for long term storage. The formulation was developed to prevent product loss, as well as minimize the purity and bioactivity decline against stresses encountered during production, storage, shipping and handling. Different buffer systems, excipients, and surfactants were evaluated.

First, a pH/buffer and excipient/surfactant combination screening study was performed to determine the optimal buffer system, excipient and surfactant strength for drug product formulation. In this study, the buffer systems evaluated included histidine buffer, acetate buffer and a mixture of these two buffers. The excipients evaluated included sucrose and trehalose·2H₂O. The surfactant and range evaluation included polysorbate 80 (PS80), 0.02%-0.06% (weight/volume (w/v)). The stress conditions evaluated included thermal stress (40° C. with incubation for up to 4 weeks), shear stress (agitation at 300 rpm at 25° C. for 3 days), and freeze-thaw stress (up to 5 freeze-thaw cycles between room temperature and −70° C.). Overall, 20 mM histidine buffer pH 6.0 was chosen as the optimal buffer system. Trehalose·2H₂O at the concentration of 8.8% (w/v) was chosen as the potential excipient and polysorbate 80 at the concentration of 0.06% (w/v) were chosen as the potential surfactant strength for AI-061 formulation. However, significant decline of PS80 content and CEX main peaks (representing both protein components of the formulation) were observed in this candidate formulation under thermal stress conditions, and a further study was performed to address this issue.

An antioxidant screening study was performed to evaluate whether adding an antioxidant could prevent the degradation of PS80 and decline of CEX main peaks. The buffer systems re-evaluated included histidine buffer and acetate buffer. The excipient and surfactant re-evaluated were 8.8% (w/v) trehalose-2H₂O and 0.06% (w/v) PS80, respectively. The antioxidants evaluated included 10 mM L-methionine and 0.2 mM EDTA·2Na·2H₂O. The stress conditions evaluated included thermal stress (25° C. and 40° C. for up to 4 weeks). Based on the antioxidant screening study, 0.2 mM EDTA·2Na·2H₂O was chosen as the optimal antioxidant for the AI-061 formulation.

Finally, a formulation confirmation study was performed to determine the optimal drug product formulation. AI-025 protein with a target concentration of 10 mg/mL mixed with ONC-392 protein with a target concentration of 10 mg/mL were prepared in 20 mM histidine buffer with 8.8% (w/v) trehalose·2H₂O, 0.2 mM EDTA·2Na·2H₂O, 0.06% (w/v) PS80 at pH 6.0. Samples were then evaluated for the stability under different temperature conditions (40° C. for up to 4 weeks, 25° C. for up to 3 months, 2-8° C. for up to 3 months), agitation at 25° C. with 100 rpm for up to 3 days, and freeze-thaw cycling for up to 5 cycles between room temperature and −70° C. The study results demonstrated drug product stability and compatibility with the planned container closure system.

Based on the formulation screening study results and a confirmation study, 10 mg/mL AI-025B-DS, 10 mg/mL ONC-392 in 20 mM histidine buffer with 8.8% (w/v) trehalose·2H₂O, 0.2 mM EDTA·2Na·2H₂O, 0.06% (w/v) PS80 at pH 6.0 was chosen as the final clinical formulation for the AI-061 at 2-8° C. storage. The excipients and their stabilizing role used in the formulation of AI-061 are summarized in Table 3.

TABLE 3
Role of Excipients in the Drug Product Formulation

| Excipient | Contribution to Stability |
|---|---|
| L-histidine | Buffer component |
| L-histidine monohydrochloride monohydrate | Buffer component |
| D(+)-Trehalose Dihydrate | Stabilizing agent |
| EDTA•2Na•2H₂O | Antioxidant |
| Polysorbate 80 | Surfactant |
| Water for Injection | Solvent |

The batch formula on a unit basis and for a 22 L drug product (DP) clinical batch is summarized in Table 4 below. The actual batch size may vary according to the clinical supply demand.

TABLE 4
Drug Product Batch Formula

| | | | | Quantity | | |
|---|---|---|---|---|---|---|
| | | Quantity | | per Batch | | |
| Component | Quality Standard | per mL | | (22 L) | | Function |
| AI-025B-DS | In-house specification | 10 | mg | 220 | g | Active |
| | | | | | | ingredient |
| ONC-392 | In-house specification | 10 | mg | 220 | g | Active |
| | | | | | | ingredient |
| L-histidine | Ph. Eur., USP, JP | 1.515 | mg | 33.33 | g | Buffer |
| | | | | | | component |
| L-histidine | Ph. Eur. | 2.146 | mg | 47.212 | g | Buffer |
| monohydrochloride | | | | | | component |
| D(+)-Trehalose | Ph. Eur., USP, JP | 88 | mg | 1936 | g | Stabilizing |
| Dihydrate | | | | | | agent |
| EDTA•2Na•2H2O | Ph. Eur., USP | 0.074 | mg | 1.628 | g | Antioxidant |
| Polysorbate 80 | USP, Ph. Eur., JP | 0.6 | mg | 13.2 | g | Surfactant |

| | | | | |
|---|---|---|---|---|
| Water for Injection | USP/Ph. Eur. | q.s. to 1 | q.s. to 22 | Solvent |
| | | mL | L | |

### Example 3

### Formulation Characterization

This example provides a characterization of a formulation of an antibody composition provided herein. AI-061 is a co-formulated drug product (DP) containing two drug substances: ONC-392 drug substance and AI-025B-DS, with the target protein concentration ratio of ONC-392 mAb and AI-025B mAb fixed at 1:1. An extended characterization study was performed to evaluate whether there are molecular (higher-order structure) and biological interactions between ONC-392 and AI-025B in AI-061 DP. The purpose of the characterization is three-fold, see Table 5 below.

TABLE 5
Purpose of the characterization of ONC-392, AI-025B-DS and AI-061

| Items | Content | Formulation |
|---|---|---|
| Purpose 1 | For ONC-392, to determine if the | Formulation 1 (ONC-392 DS |
| | thermal stability and the secondary and | buffer system): 20 mM |
| | tertiary structures will be impacted by | histidine buffer, 8.8% (w/v) α, |
| | different formulation systems | α--trehalose dihydrate, and |
| | (formulation 1 and 2). | 0.06% (w/v) polysorbate 80, |
| | | pH 6.0 |
| | | Formulation 2 (AI-061 buffer): |
| | | 20 mM histidine buffer with |
| | | 8.8% (w/v) trehalose•2H₂O, |
| | | 0.2 mM Disodium |
| | | Ethylenediaminetetraacetate |
| | | (EDTA) Dihydrate, 0.06% |
| | | (w/v) PS80 at pH 6.0 |
| Purpose 2 | For AI-025B, to determine if the | Formulation 1 (WBP2448 DS |
| | thermal stability and the secondary and | buffer system): 20 mM NaAC- |
| | tertiary structures will be impacted by | HAC, 9% (w/v) Sucrose, |
| | different formulation systems | 0.02% (w/v) PS80, pH 5.8. |
| | (formulation 1 and 2). | Formulation 2: (AI-061 buffer |
| | | system): 20 mM histidine |
| | | buffer with 8.8% (w/v) |
| | | trehalose•2H₂O, 0.2 mM |
| | | Disodium |
| | | Ethylenediaminetetraacetate |
| | | (EDTA) Dihydrate, 0.06% |
| | | (w/v) PS80 at pH 6.0 |
| Purpose 3 | For the co-formulation product, to | AI-061 buffer system: 20 mM |
| | determine if there are interactions | histidine buffer with 8.8% |
| | between ONC-392 and AI-025B in | (w/v) trehalose•2H₂O, 0.2 mM |
| | both non-clinical and clinical DP | Disodium |
| | batches. | Ethylenediaminetetraacetate |
| | | (EDTA) Dihydrate, 0.06% |
| | | (w/v) PS80 at pH 6.0 |

For purpose 3, AI-061 was characterized by a panel of methods including thermal stability analysis by differential scanning calorimetry (DSC), higher-order structure determination by ultraviolet-circular dichroism (UV CD), particle size and distribution analysis by differential light scattering (DLS), Cell Based Potency Assay for ONC-392 and Cell Based Potency Assay for AI-025B. By comparing the characterization results of AI-061 with the characterization results of the single ONC-392 and AI-025B molecules, it was possible evaluate whether there was any interaction that may affect the safety or efficacy.

For purpose 1 and 2, the samples of ONC-392 and AI-025B presented in Table 6 were used.

TABLE 6
Information of the DS lots used in the process development characterization studies

| | Sample Name | | | |
|---|---|---|---|---|
| | | ONC-392 with | | |
| | ONC-392 | AI-061 buffer | AI-025B-DS | WBP2448 DS |
| Batch Source | Clinical Lot | NA | Clinical Lot | Clinical Lot |
| Purpose | Purpose 1 | Purpose 1 | Purpose 2 | Purpose 2 |
| | | Purpose 3 | Purpose 3 | |
| Lot No. | 0000065775 | 0000065775-CO | 25722201 | 24482201 |
| Protein | 30.0 mg/mL | 29.7 mg/mL | 20.1 mg/mL | 19.9 mg/mL |
| Concentration | | | | |
| Storage | −40° C. ± 5° C. | −70° C. ± 10° C. | −70° C. ± 10° C. | −70° C. ± 10° C. |
| Condition | | | | |
| Formulation | 20 mM Histidine | 20 mM Histidine buffer, 8.8% (w/v) | | 20 mM NaAC- |
| Buffer | buffer, 8.8% | Trehalose•2H₂O, 0.06% (w/v) PS80, | | HAC, 9% (w/v) |
| | (w/v) | 0.2 mM EDTA•2Na•2H₂O, pH 6.0 | | Sucrose, 0.02% |
| | Trehalose•2H₂O, | | | (w/v) PS80, pH |
| | 0.06% (w/v) | | | 5.8. |
| | PS80, pH 6.0 | | | |

| | |
|---|---|
| Comments | WBP2448 DS and AI-025B-DS went through the 1^{st} UF/DF process together, |
| | then AI-025B-DS is further processed through the 2^{nd} UF/DF into AI-061 buffer |
| | (refer to Section 3.2.S.2.2). |
| | ONC-392 DS with AI-061 buffer is prepared by mixing ONC-392 DS (Lot No.: |
| | 0000065775) and AI-061 EDTA Stock Solution (20 mM Histidine buffer, 8.8% |
| | (w/v) Trehalose•2H₂O, 0.06% (w/v) PS80, 20 mM EDTA•2Na•2H₂O, pH 5.8) |
| | per the volume ratio of 99:1. The protein concentration is the theoretical value, |
| | which was based on the volume. To distinguish from the Lot number of ONC- |
| | 392 DS, the Lot number assigned to ONC-392 with AI-061 buffer is |
| | 0000065775-CO. |

Regarding purpose 3, in addition to the single protein samples in Table 6, samples of the non-clinical and clinical batches of AI-061 listed in Table 7 were used. The characterization results are summarized in Table 8. The results showed that there were no significant interactions between ONC-392 and AI-025B in the co-formulated AI-061 at molecular (higher-order structure) and biological levels.

TABLE 7
Information of the AI-061 lots used in the process development characterization studies

| Sample Name | AI-061 |
|---|---|
| Product Description | Co-formulated product with ONC-392 (Lot No.: |
| | 0000065775) and AI-025B (Lot No.: 25722201). |
| Lot Number | Clinical batch: 20221202 |
| | Non-clinical batch: 20221101 |
| Strength | 200 mg/10 mL |
| Total Protein Concentration (Target) | 20.0 mg/mL |
| Protein Concentration of ONC-392 | 10.0 mg/mL |
| (Target) | |
| Protein Concentration of AI-025B | 10.0 mg/mL |
| (Target) | |
| Ratio of Protein Concentration between | 1.00 |
| ONC-392 and AI-025B (Target) | |
| Formulation | 20 mM Histidine buffer, 8.8% (w/v) Trehalose•2H₂O, |
| | 0.06% (w/v) PS80, 0.2 mM EDTA•2Na•2H₂O, pH 6.0 |
| Storage Condition: | 2-8° C., protected from light |
| Shipping Instructions: | 5 ± 3° C. |

TABLE 8
Summary of characterization study results

| Purpose | Test Item | Results | Conclusion |
|---|---|---|---|
| To assess | Thermal | The absolute difference in T_{Onset}, Tₘ₁, | Purpose 1: |
| structure | Stability | Tₘ₂, Tₘ₃ and Tₘ₄ of ONC-392 DS | The thermal stability of ONC- |
| and | Analysis by | with AI-061 buffer and ONC-392 DS | 392 DS with AI-061 buffer |
| thermal | Differential | were no more than 3.0° C. and the | and ONC-392 DS were |
| stability | Scanning | shape of the profiles was visually | comparable. |
| | Calorimetry | comparable. | |
| | (DSC) | For AI-025B DS and WBP2448 DS, | Purpose 2: |
| | | the absolute difference in T_{Onset}, and | The thermal stability of AI- |
| | | Tₘ₁ were 3.3 and 3.0° C. respectively, | 025B in different formulation |
| | | and the absolute differences in Tₘ₂, | buffer had no or minor |
| | | Tₘ₃ were no more than 3.0° C., | difference. |
| | | meanwhile the shape of profiles was | |
| | | visually comparable. | |
| | | The absolute differences in T_{Onset}, | Purpose 3: |
| | | Tₘ₁, Tₘ₂, Tₘ₃, Tₘ₄ and Tₘ₅ between | The thermal stability of AI- |
| | | AI-061 non clinical DP and AI-061 | 061 non-clinical and clinical |
| | | clinical DP were no more than 3.0° C., | DP were comparable, and no |
| | | and the shape of profiles of AI-061 | interaction was observed |
| | | non clinical DP, AI-061 clinical DP | between ONC-392 and AI- |
| | | and the reconstructed theoretical | 025B in both AI-061 non- |
| | | profile were visually comparable. | clinical and clinical DP |
| To assess | Higher-order | No significant difference was | Purpose 1: |
| secondary | Structure | observed in the far-and near-UV CD | The secondary and tertiary |
| and | Determination | spectra between ONC-392 DS and | structures of ONC-392 |
| tertiary | by Ultraviolet- | ONC-392 DS with AI-061 buffer. | protein between ONC-392 DS |
| structure | Circular | | and ONC-392 DS with AI- |
| | Dichroism (UV | | 061 buffer were consistent. |
| | CD) | 2. No significant difference was | Purpose 2: |
| | | observed in the far-and near-UV CD | The secondary and tertiary |
| | | spectra between AI-025B DS and | structures of AI-025B protein |
| | | WBP2448 DS, | between AI-025B DS and |
| | | | WBP2448 DS were |
| | | | consistent. |
| | | 3. No significant difference was | Purpose 3: |
| | | observed in the far-and near-UV CD | The secondary and tertiary |
| | | spectra between AI-061 non clinical | structures between AI-061 |
| | | DP, AI-061 clinical DP and | non-clinical and clinical DP |
| | | reconstructed profile of AI-061. | were consistent, and no |
| | | | interaction was observed |
| | | | between ONC-392 and AI- |
| | | | 025B in AI-061 non-clinical |
| | | | and clinical DP. |
| To assess | Particle Size and | The experimental average diameter of | Purpose 3: No significant |
| colloidal | Distribution | AI-061 was close to the theoretical | interactions were observed |
| stability | Analysis by | average diameter of monomer and not | between ONC-392 and AI- |
| and cross- | Differential | in the range of the theoretical average | 025B in AI-061 non-clinical |
| interactions, | Light Scattering | diameter of the AI-061 as dimer, | and clinical DP. |
| aggregates | (DLS) | indicating that there was no protein- | |
| | | protein correlation. | |
| To assess | Potency (Cell | AI-061 (non-clinical and clinical DP) | Purpose 3: |
| non- | Based Assay for | and AI-025B-DS showed similar | There were no obvious |
| competitive | AI-025B) | dose-dependent curves and their | biological (cell-based |
| binding | | relative potencies were comparable, | potency) interactions between |
| to the | | while ONC-392 DS (with AI-061 | ONC-392 and AI-025B in the |
| target | | buffer) showed no dose-dependent | co-formulated AI-061. |
| | | curve, indicating that ONC-392 had | |
| | | no impact on AI-025B cell-based | |
| | | potency. | |
| | Potency (Cell | AI-061 (non-clinical DP and clinical | |
| | Based Assay for | DP) and individual ONC-392 DS | |
| | ONC-392) | (with AI-061 buffer) showed similar | |
| | | dose-dependent curves and their | |
| | | relative potencies were comparable, | |
| | | while AI-025B-DS showed no dose- | |
| | | dependent curve, indicating that AI- | |
| | | 025B had no impact on ONC-392 | |
| | | cell-based potency. | |

### Example 4

### Formulation

This example describes a formulation for an antibody composition disclosed herein. The ONC-392 drug substance formulation comprises 20 mM histidine buffer, 8.8% (w/v) α, α-trehalose dihydrate, and 0.06% (w/v) polysorbate 80, pH 6.0, whereas the AI-025 drug substance (AI-025B-DS) formulation comprises 20 mM NaAC-HAC, 9% (w/v) Sucrose, 0.02% (w/v) PS80, pH 5.8. Accordingly, the two drug substances must be mixed and formulated into the co-formulation buffer (AI-061 formulation buffer) at the correct concentration and ratios.

The drug product manufacturing process consists of AI-025 drug substance thawing, ONC-392 drug substance thawing, diluent preparation, EDTA 2Na·2H₂O stock solution preparation, pooling and mixing, and sterile filtration, prior to aseptic filling, stoppering, capping, visual inspection, bulk packaging, and storage at 2-8° C. The nominal protein concentration of AI-025B-DS and ONC-392 drug substance are 20.0 g/L and 30.0 g/L, respectively, and the protein concentration of drug product after mixing is 20 mg/mL. The concentration of EDTA 2Na 2H₂O in drug product is 0.2 mM. Drug product at 20 mg/mL is 0.22 μm sterile filtered and filled into 10 mL glass vials. The target fill volume is 10.73 mL to allow an extractable volume of 10 mL/vial.

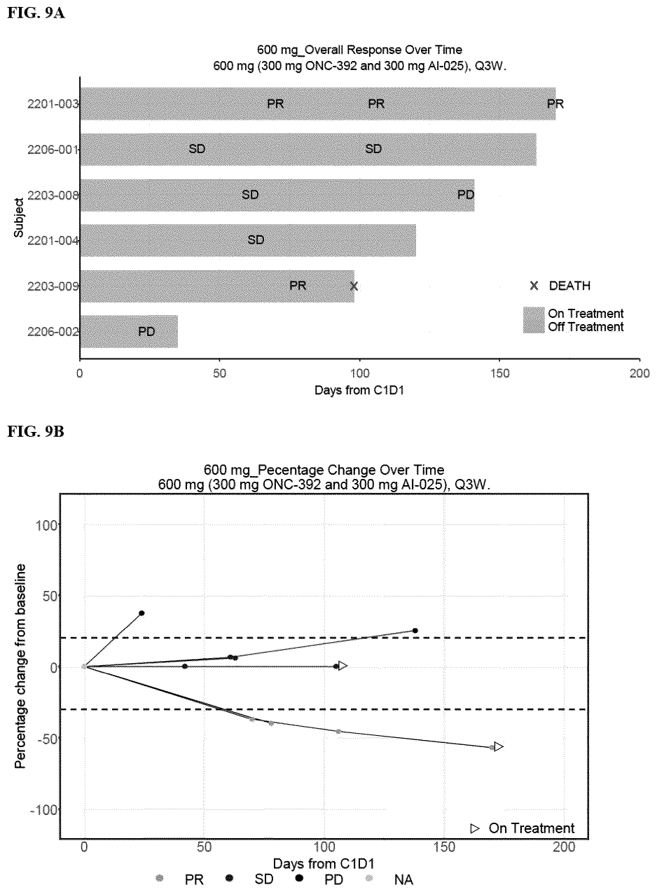

The drug product manufacturing process flow is shown in FIG. 7. In-process testing is performed at critical steps in the drug product manufacturing process. Prior to sterile filtration several parameters such as pH, osmolality, total protein concentration, ratio of protein concentrations, EDTA·2Na·2H₂O concentration, bioburden and endotoxin are tested.

### Example 5

### Clinical Trial on the Efficacy of Anti-CTLA-4/Anti-PD-1 Antibody Combination Therapy for Treating Cancer

This example demonstrates that combined anti-CTLA-4/anti-PD-1 antibody therapy disclosed herein effectively treats cancer. The drug product was AI-061 as disclosed herein, in which ONC-392 (anti-CTLA-4 antibody) and AI-025 (anti-PD-1 antibody) are present in a 1:1 ratio. The trial was a phase 1 dose escalation study. The subjects had to meet the following inclusion criteria: adult patients with histology confirmed solid tumors; disease progression after systemic treatment; have RECIST 1.1 measurable lesions; ECOG 0/1; adequate organ functions; and lactic dehydrogenase (LDH)≤upper limit of normal (ULN).

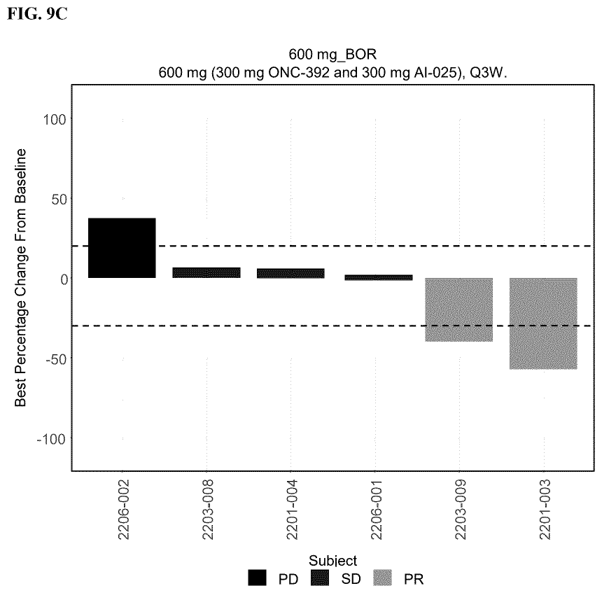

A total of 18 subjects were enrolled from July 2023 to February 2024. Three fixed dose regimens were investigated: 200 mg, every three weeks (Q3W) (n=3); 400 mg, Q3W (n=9); 600 mg, Q3W (n=6) (FIG. 8). The subjects had the following tumor types: adenocystic carcinoma (1), cervical cancer (2), cholangiocarcinoma (2), colorectal cancer (1), gastroesophageal adenocarcinoma (1), hepatocellular carcinoma (1), melanoma (1), NSCLC (4), ovarian cancer (1), pancreatic cancer (2), renal cell carcinoma (2). No dose limiting toxicities (DLT) were observed. The patients' characteristics are shown in the table below.

TABLE 9

| | AI-061 | AI-061 | AI-061 | |
|---|---|---|---|---|
| Characteristic | 200 mg | 400 mg | 600 mg | Total |
| Numbers | N = 3 | N = 9 | N = 6 | N = 18 |

Age

| | | | | |
|---|---|---|---|---|
| Median (range) - yr | 68.0 | 62.0 | 61.5 | 62.5 |
| | (35, 68) | (58, 80) | (57, 74) | (35, 80) |

Sex - no. (%)

| | | | | |
|---|---|---|---|---|
| Male | 2 | 4 | 2 | 8 (44.4) |
| Female | 1 | 5 | 4 | 10 (55.6) |
| Race | | | | |
| White | 3 | 7 | 6 | 16 (88.9) |
| Other | 0 | 2 | 0 | 2 (11.1) |

ECOG performance-status score - no. (%)

| | | | | |
|---|---|---|---|---|
| 0 | 1 | 5 | 2 | 8 (44.4) |
| 1 | 2 | 4 | 4 | 10 (55.6) |

Number of Prior lines of Cancer Tx

| | | | | |
|---|---|---|---|---|
| 0 | 0 | 1 | 0 | 1 (5.6) |
| 1-2 | 0 | 1 | 1 | 2 (11.1) |
| 3-5 | 0 | 2 | 2 | 4 (22.2) |
| >5 | 3 | 5 | 3 | 11 (61.1) |

Metastatic Diseases

| | | | | |
|---|---|---|---|---|
| Yes | 3 | 7 | 6 | 16 (88.9) |
| No | 0 | 2 | 0 | 2 (11.1) |

Prior anti-PD-(L)1 or other IO

| | | | | |
|---|---|---|---|---|
| Durvalumab | 0 | 0 | 1 | 1 (5.6) |
| Ipilimumab | 1 | 0 | 1 | 2 (11.1) |
| Nivolumab | 1 | 1 | 1 | 3 (16.7) |
| Pembrolizumab | 0 | 3 | 0 | 3 (16.7) |
| Tremelimumab | 0 | 0 | 1 | 1 (5.6) |

At the data cut date on Mar. 1, 2024, there were 11 grade 3 treatment related adverse events in 7 subjects (38%). There was no grade 4 or grade 5 AEs related to study drug. A summary of the safety data is shown in the table below.

TABLE 10

| | 200 | 400 | 600 | |
|---|---|---|---|---|
| Summary of TEAEs, TRAEs, | mg | mg | mg | Total |
| irAEs, SAEs | N = 3 | N = 9 | N = 6 | N = 18 |
| Subjects with Any TEAEs | 3 | 8 | 6 | 17 (94.4) |
| Grade >=3 TEAEs | 1 | 4 | 4 | 9 (50.0) |
| Any TEAEs related to AI-061 | 2 | 5 | 6 | 13 (72.2) |
| Grade >=3 TRAEs related to | 0 | 3 | 4 | 7 (38.9) |
| AI-061 | | | | |
| SAE | 1 | 2 | 4 | 7 (38.9) |
| SAE related to AI-061 | 0 | 2 | 2 | 4 (22.2) |
| AE leading to study drug | 0 | 1 | 2 | 3 (16.6) |
| discontinuation | | | | |
| Grade >=3 irAEs | 0 | 3 | 3 | 6 (33.3) |
| Gr. 3 TRAEs related to AI-061 | | | | |
| Myocarditis | 0 | 1 | 0 | 1 (5.6) |
| Colitis | 0 | 1 | 0 | 1 (5.6) |
| Liver enzyme increased | 0 | 1 | 1 | 2 (11.1) |
| (AST/ALT/GGT) | | | | |
| Diarrhea | 0 | 0 | 2 | 2 (11.1) |
| Immune-mediated colitis | 0 | 0 | 1 | 1 (5.6) |
| Fatigue | 0 | 0 | 1 | 1 (5.6) |
| Decreased appetite | 0 | 0 | 1 | 1 (5.6) |
| Hypokalaemia | 0 | 0 | 1 | 1 (5.6) |
| Back pain | 0 | 0 | 1 | 1 (5.6) |

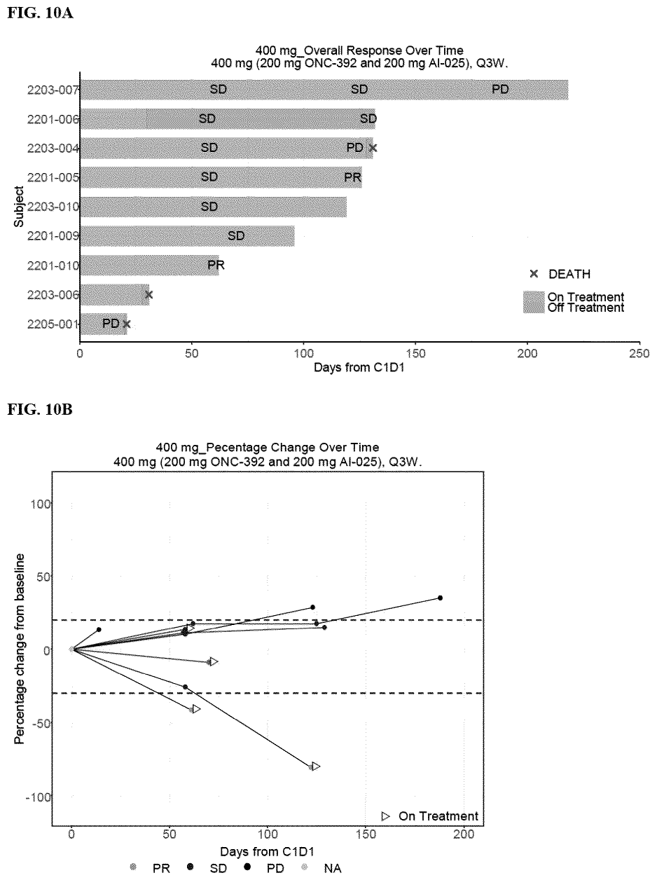

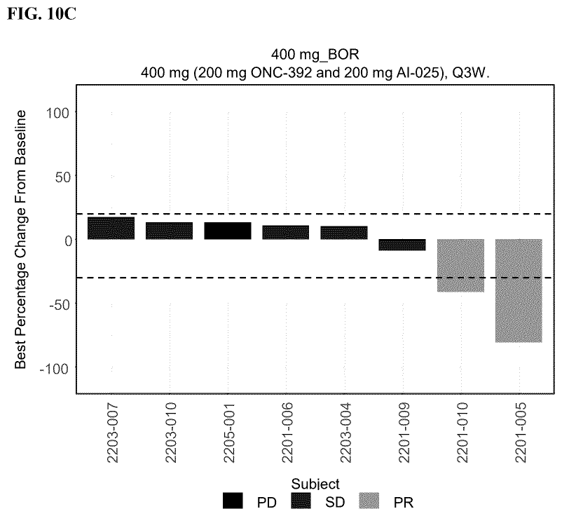

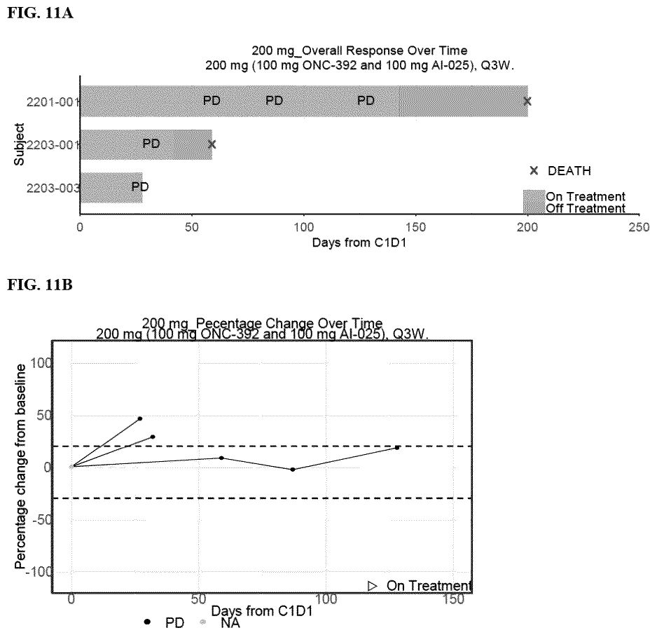

All subjects that were alive received at least one assessment for tumor responses. At the data cut date on Apr. 22, 2024, there were 2 partial responses (PR) in the 600 mg group (33%) (Table 11 and FIG. 9A-C) and 2 partial responses (PR) in the 400 mg group (22%) (Table 12 and FIG. 10A-C). There was no response in the 200 mg group (Table 13 and FIG. 11).

TABLE 11
Summary in 600 mg cohort

| Patient | Cohort | Cancer | Age/gender |
|---|---|---|---|
| A | 600 mg | metastatic cholangiocarcinoma | 65/M |
| B* | 600 mg | Adenocystic carcinoma of trachea | 60/F |
| C | 600 mg | NSCLC - Adenocarcinoma | 63/F |
| D | 600 mg | Met. CRC | 60/F |
| E* | 600 mg | Metastatic Cervical SCC | 57/F |
| F | 600 mg | Metastatic renal cell carcinoma | 74/M |

*Partial responder

TABLE 12
Summary in 400 mg cohort

| Patient | Cohort | Cancer | Age/gender |
|---|---|---|---|
| G | 400 mg | Metastatic NSCLC | 78/M |
| H | 400 mg | Metastatic Ovarian cancer | 59/F |
| I | 400 mg | Metastatic pancreatic adenocarcinoma | 58/F |
| J | 400 mg | NSCLC | 64/F |
| K | 400 mg | Cancer of Bile Duct | 62/F |
| L* | 400 mg | Metastatic Gastro Oesophageal | 58/M |
| | | Adenocarcinoma | |
| M | 400 mg | Melanoma | 59/F |
| N | 400 mg | hepatocellular carcinoma | 79/M |
| O* | 400 mg | stage IV type II papillary RCC | 80/M |

*Partial responder

TABLE 13
Summary in 200 mg cohort

| Patient | Cohort | Cancer | Age/gender |
|---|---|---|---|
| P | 200 mg | NSCLC | 68/M |
| Q | 200 mg | Cervical | 35/F |
| R | 200 mg | Pancreatic | 68/M |

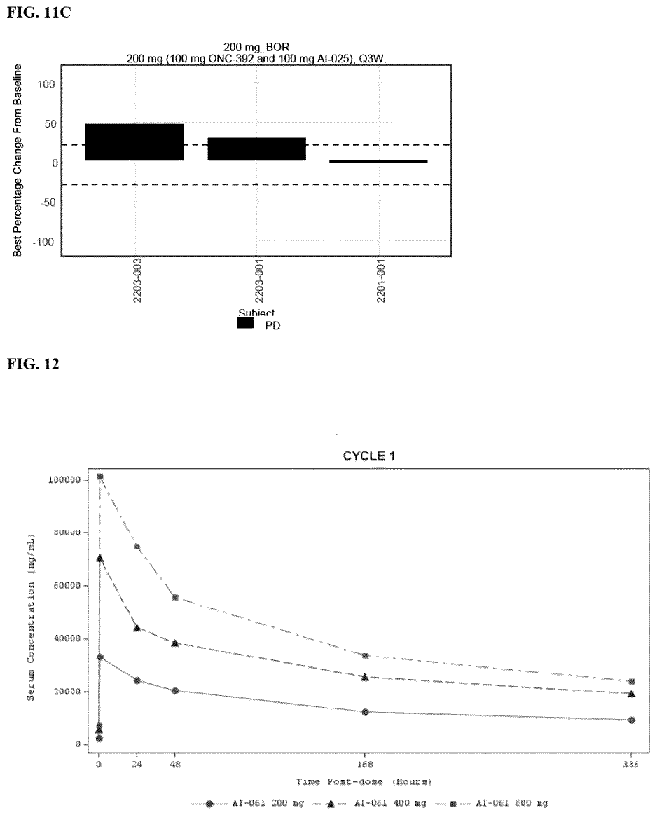

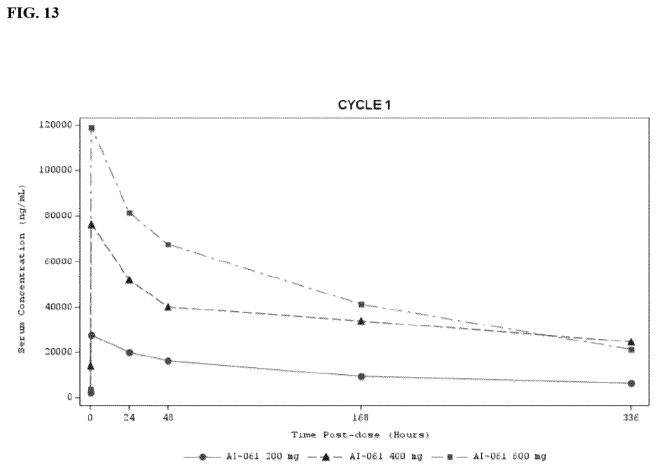

The pharmacokinetic (PK) analyses on ONC-392 (Table 14 and FIG. 12) and AI-025 (Table 15 and FIG. 13) showed the predicted pattern for stable monoclonal antibodies. The similarity of their PK profiles supports their coadministration.

TABLE 14
Summary of key pharmacokinetic parameters of ONC-
392 by cohort and period in a PK analysis set

| | | 100 mg | 200 mg | 300 mg | Total |
|---|---|---|---|---|---|
| | | AI-061 200 | AI-061 400 | AI-061 600 | Total |
| Cycle 1 | ONC392 | mg (N = 3) n | mg (N = 6) n | mg (N = 6) n | (N = 15) n |
| Parameters | Statistics | (%) | (%) | (%) | (%) |
| Tₘₐₓ (h) | Median (range) | 0.780 | 1.350 | 12.290 | 1.370 |
| | | (0.75-1.22) | (1.30-1.63) | (1.33-24.55) | (0.75-24.55) |
| Cₘₐₓ (μg/mL) | Mean (CV %) | 33.17 | 70.60 | 98.17 | 74.14 |
| | | (13.4) | (29.7) | (20.8) | (40.9) |
| AUC_{0-inf} | Mean (CV %) | 9007.71 | 20250.30 | 30603.05 | 22434.05 |
| (h*μg/mL) | | (13.6) | (25.7) | (32.8) | (50.6) |
| t_{1/2} (h) | Mean (CV %) | 290.31 | 379.18 | 434.55 | 384.23 |
| | | (1.8) | (33.3) | (54.2) | (45.5) |

TABLE 15
Summary of key pharmacokinetic parameters of AI-
025 by cohort and period in a PK analysis set

| | | 100 mg | 200 mg | 300 mg | Total |
|---|---|---|---|---|---|
| | | AI-061 200 | AI-061 400 | AI-061 600 | Total |
| Cycle 1 | AI-025 | mg (N = 3) n | mg (N = 6) n | mg (N = 6) n | (N = 15) n |
| Parameters | Statistics | (%) | (%) | (%) | (%) |
| Tₘₐₓ (h) | Median (range) | 0.780 | 1.350 | 1.510 | 1.370 |
| | | (0.75-1.22) | (1.30-1.63) | (1.33-23.92) | (0.75-23.92) |
| Cₘₐₓ (μg/mL) | Mean (CV %) | 27.63 | 76.45 | 110.23 | 80.20 |
| | | (15.3) | (38.5) | (23.3) | (48.8) |
| AUC_{0-inf} | Mean (CV %) | 6320.51 | 26535.36 | 23659.88 | 20543.25 |
| (h*μg/mL) | | (9.0) | (47.1) | (28.5) | (54.5) |
| t_{1/2} (h) | Mean (CV %) | 253.78 | 373.00 | 248.02 | 287.80 |
| | | (11.0) | (33.9) | (53.1) | (42.3) |

Accordingly, the results of the trial indicate that combined treatment with an anti-CTLA-4 antibody and an anti-PD-1 antibody disclosed herein is effective against cancer.

1. A composition comprising an anti-CTLA-4 antibody and an anti-PD-1 antibody, wherein:
(a) the anti-CTLA-4 antibody comprises (i) a light chain variable region comprising a complementarity determining region (CDR) 1 comprising the sequence set forth in SEQ ID NO: 1, a CDR2 comprising the sequence set forth in SEQ ID NO: 3, and a CDR3 comprising the sequence set forth in SEQ ID NO: 5; and (ii) a heavy chain variable region comprising a CDR1 comprising the sequence set forth in SEQ ID NO: 6, a CDR2 comprising the sequence set forth in SEQ ID NO: 9, and a CDR3 comprising the sequence set forth in SEQ ID NO: 10; and
(b) the anti-PD-1 antibody comprises (i) a light chain variable region comprising a CDR1 comprising the sequence set forth in SEQ ID NO: 25, a CDR2 comprising the sequence set forth in SEQ ID NO: 26, and a CDR3 comprising the sequence set forth in SEQ ID NO: 27; and (ii) a heavy chain variable region comprising a CDR1 comprising the sequence set forth in SEQ ID NO: 31, a CDR2 comprising the sequence set forth in SEQ ID NO: 32, and a CDR3 comprising the sequence set forth in SEQ ID NO: 33.

2. The composition of claim 1, wherein the anti-CTLA-4 antibody comprises a light chain variable region comprising the sequence set forth in SEQ ID NO: 12 and a heavy chain variable region comprising the sequence set forth in SEQ ID NO: 16.

3. The composition of claim 2, wherein the anti-CTLA-4 antibody comprises a light chain comprising the sequence set forth in SEQ ID NO: 23 and a heavy chain comprising the sequence set forth in SEQ ID NO: 21.

4. The composition of claim 1, wherein the anti-PD-1 antibody comprises a light chain variable region comprising the sequence set forth in SEQ ID NO: 28 and a heavy chain variable region comprising the sequence set forth in SEQ ID NO: 34.

5. The composition of claim 4, wherein the anti-PD-1 antibody comprises a heavy chain comprising an IgG4 comprising the sequence set forth in SEQ ID NO: 35 or 36.

6. The composition of claim 5, wherein the anti-PD-1 antibody comprises a light chain comprising the sequence set forth in SEQ ID NO: 30 and a heavy chain comprising the sequence set forth in SEQ ID NO: 37.

7. The composition of claim 1, wherein the anti-CTLA-4 antibody comprises a light chain comprising the sequence set forth in SEQ ID NO: 23 and a heavy chain comprising the sequence set forth in SEQ ID NO: 21; and the anti-PD-1 antibody comprises a light chain comprising the sequence set forth in SEQ ID NO: 30 and a heavy chain comprising the sequence set forth in SEQ ID NO: 37.

8. The composition of claim 1, wherein the composition comprises 1-50 mg/mL of each of the anti-CTLA-4 antibody and the anti-PD-1 antibody.

9. The composition of claim 8, comprising 10 mg/mL of each of the anti-CTLA-4 antibody and the anti-PD-1 antibody.

10. The composition of claim 1, wherein the composition comprises a pharmaceutically acceptable carrier.

11. The composition of claim 10, wherein the composition comprises one or more of a histidine buffer, α, α-trehalose dihydrate, polysorbate 80 (PS80), and EDTA.

12. The composition of claim 11, comprising 5-40 mM histidine buffer, 7.0-10.0% (w/v) α, α-trehalose dihydrate, and 0.01-0.10 (w/v) PS80, and wherein the pH of the composition is 5.5-6.5.

13. The composition of claim 12, comprising 20 mM histidine buffer, 8.8% (w/v) α, α-trehalose dihydrate, 0.06% (w/v) PS80, and 0.2 mM EDTA·2Na·2H₂O, and wherein the pH of the composition is 6.0.

14. A method of treating a cancer in a subject in need thereof, comprising administering to the subject the composition of claim 1.

15. The method of claim 14, wherein the cancer is a tumor.

16. The method of claim 14, wherein the cancer is selected from the group consisting of melanoma, non-small cell lung carcinoma (NSCLC), head and neck squamous cell carcinoma (HNSCC), ovarian cancer, endometrial carcinoma, cervical cancer, renal cell carcinoma, bladder cancer, esophageal cancer, gastric cancer, gastroesophageal (GE) junction cancer, colorectal cancer, anal cancer, hepatocellular carcinoma, cancer of a bile duct, adenoid cystic carcinoma (ACC), and triple negative breast cancer (TNBC).

17. The method of claim 16, wherein the ovarian cancer is high grade serous ovarian carcinoma.

18. The method of claim 17, wherein the high grade serous ovarian carcinoma is primary peritoneal cancer or fallopian tube cancer.

19. The method of claim 14, wherein the cancer is PD-1 resistant.

20. The method of claim 14, comprising administering up to 10 mg/kg of each of the anti-CTLA-4 antibody and the anti-PD-1 antibody to the subject.

21. The method of claim 14, comprising administering the composition intravenously.

22. The method of claim 14, wherein the composition is administered once every about 3 weeks.

23. A composition comprising an anti-CTLA-4 antibody and an anti-PD-1 antibody, wherein:
(a) the anti-CTLA-4 antibody comprises a light chain comprising the sequence set forth in SEQ ID NO: 23 and a heavy chain comprising the sequence set forth in SEQ ID NO: 21;
(b) the anti-PD-1 antibody comprises a light chain comprising the sequence set forth in SEQ ID NO: 30 and a heavy chain comprising the sequence set forth in SEQ ID NO: 37;
(c) the composition comprises 20 mM histidine buffer, 8.8% (w/v) α, α-trehalose dihydrate, 0.06% (w/v) PS80, and 0.2 mM EDTA·2Na·2H₂O; and
(d) the pH of the composition is 6.0.

24. The composition of claim 23, comprising 10 mg/mL of each of the anti-CTLA-4 antibody and the anti-PD-1 antibody.

25. A method of treating a cancer in a subject in need thereof, comprising administering to the subject the composition of claim 23.

26. The method of claim 25, wherein the cancer is a tumor.

27. The method of claim 25, wherein the cancer is selected from the group consisting of melanoma, non-small cell lung carcinoma (NSCLC), head and neck squamous cell carcinoma (HNSCC), ovarian cancer, endometrial carcinoma, cervical cancer, renal cell carcinoma, bladder cancer, esophageal cancer, gastric cancer, gastroesophageal (GE) junction cancer, colorectal cancer, anal cancer, hepatocellular carcinoma, cancer of a bile duct, adenoid cystic carcinoma (ACC), pancreatic cancer, and triple negative breast cancer (TNBC).

28. The method of claim 25, wherein the composition is administered intravenously.

29. The method of claim 25, comprising administering 10 mg/kg of each of the anti-CTLA-4 antibody and the anti-PD-1 antibody to the subject.

30. The method of claim 25, wherein the composition is administered once every 3 weeks.

31.-40. (canceled)
